(19) United States
(12) Patent Application Publication
(10) Pub. No.: US 2026/0158013 A1
(43) Pub. Date: Jun. 11, 2026

(54) COMPOSITIONS AND METHODS FOR THE PREVENTION AND TREATMENT OF OBESITY AND OBESITY-INDUCED DISEASE

(71) Applicant: Apogee Biotechnology Corporation, Hummelstown, PA (US)

(72) Inventors: Charles D. Smith, Hummelstown, PA (US); Lynn W. Maines, Palmyra, PA (US)

(21) Appl. No.: 19/045,439

(22) Filed: Feb. 4, 2025

Related U.S. Application Data
(60) Provisional application No. 63/679,916, filed on Aug. 6, 2024.

(51) Int. Cl. A61K 31/4409; A61K 31/337; A61K 31/357; A61K 31/4045; A61K 31/485; A61K 38/26; A61P 3/04; A61P 3/10

(52) U.S. Cl. CPC A61K 31/4409; A61K 31/337; A61K 31/357; A61K 31/4045; A61K 31/485; A61K 38/26; A61P 3/04; A61P 3/10

(57) ABSTRACT

The present disclosure relates to compositions and methods useful for the treatment and/or prevention of obesity and obesity-related diseases in a subject in need thereof. In various embodiments, the present disclosure relates to uses of compounds that are inhibitors of sphingosine kinase-2 (SphK2), dihydroceramide desaturase (DES1) and/or hexosylceramide synthase (GCS), such as opaganib, in the treatment and/or prevention of obesity and obesity-related diseases.

### CROSS-REFERENCE TO RELATED APPLICATIONS

This application claims the benefit of priority of U.S. Provisional Patent Application No. 63/679,916, filed Aug. 6, 2024, which is hereby incorporated herein by reference in its entirety.

### BACKGROUND OF THE DISCLOSURE

### 1. Field of the Disclosure

The present disclosure relates to compositions and methods useful for the treatment and/or prevention of obesity and obesity-related diseases in a subject in need thereof.

### 2. Technical Background

Metabolic disease, including obesity and diabetes, constitutes a major emerging health crisis in Western nations. Although the symptoms and clinical pathology and physiology of these conditions are well understood, the molecular mechanisms underlying the disease process have largely remained obscure.

What are needed are additional treatments for obesity and obesity-related diseases such as diabetes.

### SUMMARY OF THE DISCLOSURE

The disclosure relates to the treatment and/or prevention of obesity and obesity-related disease, including but not limited to hyperglycemia, insulin resistance, diabetes, metabolic syndrome, heart disease, gallbladder disease, high blood pressure, sleep apnea, kidney disease, stroke, fatty liver disease, high cholesterol, respiratory disease, depression, gout or infertility. Additionally, the treatments described herein can be used to suppress the appetite for food in a subject in need thereof. Additionally, treatments described herein can be used to improve glucose control and reduce fat mass in a subject in need thereof. Additionally, the treatments described herein can be used to promote the loss of body weight in a subject in need thereof. Without intending to be bound by theory, the present inventors believe that the treatments described herein work by inhibiting sphingolipid metabolism.

Thus, the present disclosure provides a variety of methods, pharmaceutical compositions and uses that find use in treatment of obesity and a variety of obesity-related disorders. In some embodiments, the compound used in the treatment inhibits the activity of sphingosine kinase-2 (SphK2), dihydroceramide desaturase (DES1) and/or hexosylceramide synthase (GCS). In some embodiments, the compound is 3-(4-chlorophenyl)-N-(pyridin-4-ylmethyl)-1-adamantanecarboxamide (also known as ABC294640 and opaganib) or a salt or solvate (e.g., hydrate) thereof.

### BRIEF DESCRIPTION OF THE DRAWINGS

FIG. 1. Effect of opaganib-treatment on body weight gain. Left Panel: Male C57BL/6J mice were given a CD and treated with either Vehicle (●) or 100 mg/kg Opaganib (▾) once daily, 5 days/week. Right Panel: Mice were given a HFD and treated with either Vehicle (●) or 100 mg/kg Opaganib (▪) once daily, 5 days/week. In each case, mice were weighed on the indicated days and the mean±SEM values are shown. For mice on the HFD, the statistical significance of differences reached *p<0.05 at Day 29, **p<0.01 at Day 43 and ***p<0.001 at Day 54.

FIG. 2. Effect of opaganib on food consumption. Food consumption was calculated twice weekly and normalized for the number of mice per cage for mice given either CD (Left Panel) or HFD (Right Panel) and treated with either Vehicle (▴, ●) or 100 mg/kg Opaganib (┌, ▪) once daily, 5 days/week. The mean values for food consumption per mouse are shown.

FIG. 3. Effect of opaganib on HFD-induced glucose intolerance. Left Panel: Glucose tolerance curves for Vehicle and Opaganib (100 mg/kg) male mice fed HFD or CD for 8 weeks. Right Panel: Values for the blood glucose concentration AUCs at 8 weeks are shown. Mean±SEM values are shown. ***p<0.001 vs. HFD-Vehicle.

FIG. 4. Effect of opaganib on body weight of HFD-induced obese mice. HFD-Vehicle and HFD-Opaganib mice were randomized to subgroups given HFD and either the original or the opposite treatment from Week 8 to 16. Left Panel: Body weights (mean SEM) of HFD-Vehicle→HFD-Vehicle and HFD-Vehicle→HFD-Opaganib are shown. Right Panel: Body weights of HFD-Opaganib→HFD-Vehicle and HFD-Opaganib→HFD-Opaganib are shown. ***p<0.001 vs. HFD-Opaganib→HFD-Vehicle on the same day.

FIG. 5. Effect of opaganib on HFD-induced glucose intolerance. Top Panels: Glucose tolerance curves at 12 and 16 weeks for HFD-Vehicle→HFD-Vehicle (●) and HFD-Vehicle→HFD-Opaganib (▾), HFD-Opaganib→HFD-Vehicle (▴) and HFD-Opaganib→HFD-Opaganib (▪) are shown. Bottom Panels: Values for the blood glucose concentration AUCs at 12 and 16 weeks are shown. Mean±SEM values are shown. *p<0.05, **p<0.01 and ***p<0.001 vs. HFD-Vehicle→HFD-Vehicle and #p<0.05 vs. CD-Vehicle.

FIG. 6. Effect of opaganib on HFD-induced elevation of HbA1c. Mice were given either CD or HFD and treated with Vehicle or 100 mg/kg Opaganib once daily, 5 days/week for 16 weeks. Blood obtained by tail prick was analyzed for HbA1c concentrations. Mean±SEM values are shown. **p<0.01 vs. CD-Vehicle mice.

FIG. 7. Effect of opaganib on fat deposition. Left Panel: Inguinal fat pad weights at 8 weeks for HFD-Vehicle (n=6) and HFD-Opaganib (n=5). Mean±SEM values are shown. **p<0.01 compared with the HFD-Vehicle group. Right Panel: Inguinal fat pad weights at 16 weeks for HFD-Vehicle→HFD-Vehicle (n=8) and HFD-Vehicle→HFD-Opaganib (n=10), HFD-Opaganib→HFD-Vehicle (n=5) and HFD-Opaganib→HFD-Opaganib (n=4) are shown. Data for mice maintained on CD and treated with Vehicle or Opaganib are also shown. Mean±SEM values are shown. **p<0.01 or ***p<0.001 compared with the HFD-Vehicle→HFD-Vehicle group.

FIG. 8. Effect of opaganib-treatment on female mouse body weight gain and glucose tolerance. Female C57BL/6J mice were given a CD or HFD and treated with either Vehicle or 100 mg/kg Opaganib once daily, 5 days/week. Left Panel: Female mice given CD and Vehicle (●), CD and opaganib (▪), HFD and Vehicle (♦) or HFD and opaganib (▾) were weighed on the indicated days and the mean±SEM values are shown. Right Panel: Glucose tolerance curves at 4 weeks are shown.

FIG. 9. Effects of opaganib and semaglutide on body weight and glucose tolerance in obese mice. Panel A: Male C57BL/6J mice were given HFD for 18 weeks and the treated with either Vehicle (●, n=8), opaganib (▪, n=6), semaglutide (▴, n=6) or opaganib plus semaglutide (♦, n=7). Mice were weighed on the indicated days and the mean±SEM values are shown. Panel B: Semaglutide-treated mice were randomized on Day 23 (Arrow) to Vehicle (●) or opaganib (▪) and weights were measured until Day 27. Panel C: Glucose tolerance curves at 2 weeks of treatment are shown. Mean±SEM values are shown. Panel D: Values for the blood glucose concentration AUCs at 2 weeks are shown. ****p<0.001 vs. Vehicle.

### DETAILED DESCRIPTION

The present disclosure provides a variety of treatments of obesity and obesity-induced diseases. More specifically, the present disclosure provides methods, compositions and uses for of compounds and pharmaceutical compositions for the treatment and/or prevention of obesity and obesity-related disease, including but not limited to hyperglycemia, insulin resistance, diabetes, metabolic syndrome, heart disease, gallbladder disease, high blood pressure, sleep apnea, kidney disease, stroke, fatty liver disease, high cholesterol, respiratory disease, depression, gout or infertility. Additionally, the treatments can be used to suppress the appetite for food in a subject in need thereof. Additionally, the treatments can be used to improve glucose control and reduce fat mass in a subject in need thereof. Additionally, the treatments described herein can be used to promote the loss of body weight in a subject in need thereof.

Various particular embodiments of the disclosure are provided in the claims below, which can be combined in any combination and in any number that is not technically or logically inconsistent.

Sphingolipids, a lipid class with both signaling and structural properties, have recently emerged as key players in most major tissues affected by diabetes and are essential components in the molecular etiology of this disease. Sphingosine kinases (SphKs) are enzymes that catalyze the phosphorylation of sphingosine to form sphingosine 1-phosphate (S1P). S1P is a bioactive sphingolipid involved in various cellular processes such as cell proliferation, survival, migration, and inflammation.

The present inventors have noted that, in the context of diabetes and obesity, the roles of sphingosine kinases are multifaceted, including insulin resistance, n-cell disruption, adipocyte function, inflammation and immune regulation, vascular complications and energy metabolism. Overall, the dysregulation of SphKs and the resulting alterations in sphingolipid metabolism, particularly the accumulation of S1P, can contribute to the pathogenesis of obesity and diabetes. Thus, the present inventors note that targeting sphingolipid metabolism pathways, particularly SphKs, may offer therapeutic strategies for managing diabetes and obesity and their associated complications.

The compounds and pharmaceutical compositions to be used in the treatments of the disclosure can be used in various protocols for treating animals, including humans. In one embodiment of the methods, compositions and uses of the present disclosure, SphK2 in target cells or tissues in an animal is inhibited by administering to the animal a pharmaceutical composition containing a SphK2 inhibitor. In another embodiment of the methods, compositions and uses of the present disclosure, DES1 in target cells or tissues in an animal is inhibited by administering to the animal a pharmaceutical composition containing a DES1 inhibitor. In one embodiment of the methods, compositions and uses of the present disclosure, GCS in target cells or tissues in an animal is inhibited by administering to the animal a pharmaceutical composition containing a GCS inhibitor. Inhibition of these enzymes by opaganib is described in, e.g., K. J. French et al., “Pharmacology and Antitumor Activity of ABC294640, a selective inhibitor of Sphingosine Kinase-2,” J. Pharmacology & Expt'l Therapeutics, 333(1), 129-139 (2010) and L. W. Maines et al., “Opaganib Downregulates N-Myc Expression and Suppresses In Vitro and In Vivo Growth of Neuroblastoma Cells,” Cancers (Basel), 16(9), 1779 (May 5, 2024), each of which is hereby incorporated herein by reference in its entirety.

In preferred embodiments, the methods comprise administering to a subject in need thereof a pharmaceutical composition containing an effective amount 3-(4-chlorophenyl)-N-(pyridin-4-ylmethyl)-1-adamantanecarboxamide (also known as ABC294640 and opaganib) or a salt or hydrate thereof. But, as described below, a variety of other compounds can be used.

In a particularly preferred embodiment of the use of the methods, compositions and uses of the present disclosure, the compounds or compositions can be used for treating obesity in a patient requiring such treatment, by administering the compound or composition to a patient in an amount effective to inhibit the activity of SphK2, DES1 and/or GCS of target cells of said patient. This method would involve administering to a patient with obesity a composition in an amount effective to suppress weight gain, promote weight loss and/or to reduce body fat.

In another particularly preferred embodiment of the use of the methods, compositions and uses of the present disclosure, the compounds or compositions can be used in a method for treating diabetes in a patient requiring such treatment. The compounds or compositions can be administered in an amount effective to treat diabetes. The present inventors note that diabetic states other than insulin-dependent diabetes mellitus can be treated with the treatments described herein.

In some embodiments, this can be done by administering the compound or composition to a patient in an amount effective to inhibit the aberrant sphingolipid metabolism. This method would involve administering to the patient a compound or composition in an amount effective to inhibit SphK2, DES1 and/or GCS in target tissues.

In another particularly preferred embodiment of the use of the methods, compositions and uses of the present disclosure, the compounds or compositions can be used in a method for treating metabolic syndrome, heart disease, gallbladder disease, high blood pressure, sleep apnea, kidney disease, stroke, fatty liver disease, high cholesterol, respiratory disease, depression, gout or infertility. The compounds or compositions can be administered in an amount effective to treat the disorder.

In some embodiments, this can be done by administering the compound or composition to a patient in an amount effective to inhibit the aberrant sphingolipid metabolism. This method would involve administering to the patient a compound or composition in an amount effective to inhibit SphK2, DES1 and/or GCS in target tissues.

In another particularly preferred embodiment of the use of the methods, compositions and uses of the present disclosure, the compounds or compositions can be used in combination with other methods for treating or preventing obesity and obesity-related diseases. Such other methods include but are not limited to treatment with an additional compound or pharmaceutical agent that also treat obesity by inducing weight loss. Such agents include but are not limited to semaglutide, bupropion-naltrexone, liraglutide, orlistat, phentermine-topiramate, setmelanotide, exenatide, dulaglutide and tirzepatide.

In view of the beneficial effect of inhibiting SphK2, DES1 and GCS, it is anticipated that the methods, compositions and uses of the present disclosure will be useful not only for therapeutic treatment following the onset of disease, but also for the prevention of disease in animals, including humans. The methods described herein will be essentially the same whether the compounds or pharmaceutical compositions are being administered for the treatment or prevention of disease.

While the methods, compositions and uses of the disclosure have been particularly shown and described with reference to preferred embodiments thereof, it will be understood by those skilled in the art that various alterations in form and detail may be made therein without departing from the spirit and scope of the disclosure. In particular, the specific method of use of the sphingolipid inhibitory compounds and compositions can vary significantly without departing from the discovered methods. Additionally, methods for the treatment of additional diseases that involve undesired activation of SphK2, DES1 and/or GCS within particular cells of the subject are considered to be within the scope of the following embodiments and claims.

As used herein, the term “agent” refers to a compound having a pharmacological activity—an effect of the agent on an individual. The terms “agent,” “compound,” and “drug” are used interchangeably herein.

A “patient” or an “individual” refers to any animal, such as a primate. In an embodiment, the primate is a non-human primate. In an embodiment, the primate is a human primate. Any animal can be treated using the methods and composition of the present disclosure.

As used herein, the term “synergistic effect” refers to the coordinated or correlated action of two or more agents of the present disclosure so that the combined action is greater than the sum of each acting separately. In an embodiment, agents of the present disclosure, when administered together as part of a treatment regimen, provide a therapeutic synergy without accompanying synergistic side effects (e.g., but not limited to, cross-reacting agents).

As used herein, the term “treat” is meant to administer one or more agents of the present disclosure to reduce or suppress weight gain and/or improve or normalize glucose tolerance in a patient and/or reduced at least one symptom in pathologies associated with obesity and/or diabetes.

By an “effective amount” is meant the amount of an agent or agents of the present disclosure, alone or in combination with another therapeutic regimen, required to treat a patient in need thereof in a clinically relevant manner. A sufficient effective amount of an agent or agents used to practice the present disclosure for therapeutic treatment of conditions such as obesity and/or diabetes varies depending upon the manner of administration, the age, body weight, and general health of the patient. Ultimately, the prescribers will decide the appropriate amount and dosage regimen. In a combination therapy of the disclosure, the effective amount of an agent may be less than the effective amount if the agent were administered in a non-combinatorial (single-agent) therapy. Additionally, an effective amount may be an amount of an agent in a combination therapy of the disclosure that is safe and efficacious in the treatment of pathologies associated with obesity and/or diabetes.

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

The term “minimize” or “reduce,” or a derivative thereof, includes a complete or partial inhibition of a specified biological effect (which is apparent from the context in which the term minimize is used).

As used herein, the term “a suitable period of time” refers to the period of time starting when a patient begins treatment using a method of the present disclosure, throughout the treatment, and up until when the patient stops treatment due to a reduction in symptoms associated obesity and diabetes. In an embodiment, a suitable period of time is one (1) day to (1) week. In an embodiment, a suitable period of time is one (1) week. In an embodiment, a suitable period of time is between one (1) week and two (2) weeks. In an embodiment, a suitable period of time is two (2) weeks. In an embodiment, a suitable period of time is between two (2) weeks and three (3) weeks. In an embodiment, a suitable period of time is three (3) weeks. In an embodiment, a suitable period of time is between three (3) weeks and four (4) weeks. In an embodiment, a suitable period of time is four (4) weeks. In an embodiment, a suitable period of time is between four (4) weeks and five (5) weeks. In an embodiment, a suitable period of time is five (5) weeks. In an embodiment, a suitable period of time is between five (5) weeks and six (6) weeks. In an embodiment, a suitable period of time is six (6) weeks. In an embodiment, a suitable period of time is between six (6) weeks and seven (7) weeks. In an embodiment, a suitable period of time is seven (7) weeks. In an embodiment, a suitable period of time is between seven (7) weeks and eight (8) weeks. In an embodiment, a suitable period of time is eight (8) weeks or more.

“Concurrently” means (1) simultaneously in time, or (2) at different times during the course of a common treatment schedule.

“Sequentially” refers to the administration of one active agent used in the method followed by administration of another active agent. After administration of one active agent, the next active agent can be administered substantially immediately after the first, or the next active agent can be administered after an effective time period after the first active agent; the effective time period is the amount of time given for realization of maximum benefit from the administration of the first active agent.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes, by administration of separate pills or capsules, or by separate infusions. In general, during alternation therapy, an effective dosage of each active ingredient is administered sequentially, i.e., serially, whereas in combination therapy, effective dosages of two or more active ingredients are administered together.

The term “in vitro” as used herein refers to procedures performed in an artificial environment, such as for example, without limitation, in a test tube or cell culture system. The skilled artisan will understand that, for example, an isolate SK enzyme may be contacted with a modulator in an in vitro environment. Alternatively, an isolated cell may be contacted with a modulator in an in vitro environment.

The term “in vivo” as used herein refers to procedures performed within a living organism such as, without limitation, a human, monkey, mouse, rat, rabbit, bovine, equine, porcine, canine, feline, or primate.

The term “alkyl”, as used herein alone or as part of a larger moiety, refers to a saturated acyclic aliphatic hydrocarbon, including straight chain or branched chain groups. Examples of alkyl groups include methyl, ethyl, propyl, isopropyl, butyl, iso-, sec- and tert-butyl, pentyl, hexyl, heptyl, 3-ethylbutyl, and the like. In various embodiments, an “alkyl” group has 1 to 20 carbon atoms (whenever a numerical range, e.g. “1-20”, is stated herein, it means that the group, in this case the alkyl group, may contain 1 carbon atom, 2 carbon atoms, 3 carbon atoms, etc. up to and including 20 carbon atoms). In various embodiments, an “alkyl” group has 1-6 carbon atoms. In various embodiments, an “alkyl” group has 1-4 carbon atoms, or 1-2 carbon atoms. When an “alkyl” is provided as a divalent moiety (e.g., “-alkyl-”) it is understood that the points of attachment can be on the same carbon or on different carbons.

The term “alkoxy”, as used herein alone or as part of a larger moiety, represents an alkyl group of indicated number of carbon atoms attached to the parent molecular moiety through an oxygen bridge. When a number of carbon atoms is not indicated, in various embodiments the alkoxy group has 1-6 carbon atoms, e.g., 1-4 carbon atoms, or 1-2 carbon atoms. Examples of alkoxy groups include, for example, methoxy, ethoxy, propoxy and isopropoxy. Alkoxy radicals may be further substituted with one or more halo atoms, such as fluoro, chloro or bromo, to provide “haloalkoxy” radicals. Examples of such radicals include fluoromethoxy, chloromethoxy, trifluoromethoxy, and fluoroethoxy.

The term “aryl”, as used herein alone or as part of a larger moiety, refers to an aromatic hydrocarbon ring system containing at least one aromatic ring. The aromatic ring may optionally be fused or otherwise attached to other aromatic hydrocarbon rings or non-aromatic hydrocarbon rings. Additionally, unless otherwise specified the aryl group may be optionally substituted by various groups such as halo, hydroxy, alkyl, haloalkyl, alkoxy, nitro, cyano, alkylamine, carboxy or alkoxycarbonyl. Preferred aryl group substituents include halo, alkyl, haloalkyl, hydroxy and alkoxy. Examples of aryl groups include, for example, phenyl, naphthyl, 1,2,3,4-tetrahydronaphthalene, benzodioxole, and biphenyl. Preferred examples of unsubstituted aryl groups include phenyl and biphenyl.

The term “heterocycloalkyl”, as used herein alone or as part of a larger moiety, refers to a non-aromatic ring system containing at least one heteroatom selected from nitrogen, oxygen, and sulfur. The heterocycloalkyl ring may be optionally fused to or otherwise attached to other heterocycloalkyl rings and/or non-aromatic hydrocarbon rings. Preferred heterocycloalkyl groups have from 3 to 7 members and include 1-4 heteroatoms selected from O, N and S. Examples of heterocycloalkyl groups include, for example, piperazine, morpholine, piperidine, tetrahydrofuran, pyrrolidine, and pyrazole. Preferred monocyclic heterocycloalkyl groups include piperidyl, piperazinyl, morpholinyl, pyrrolidinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like. Heterocycloalkyl radicals may also be partially unsaturated. Examples of such groups include dihydrothienyl, dihydropyranyl, dihydrofuryl, and dihydrothiazolyl.

The term “heteroaryl”, as used herein alone or as part of a larger moiety, refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. In various embodiments, the heteroaryl group is a 5-6-membered monocyclic heteroaryl having 1-3 heteroatoms selected from O, N and S. In various embodiments, the heteroaryl group is an 8-10-membered bicyclic heteroaryl having 1-5 heteroatoms selected from O, N and S. Additionally, the heteroaryl group may be optionally substituted at one or more atoms of the ring system, or may contain one or more oxo groups. Examples of heteroaryl groups include, for example, pyridine, furan, thiophene, carbazole and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, benzopyrazolyl, purinyl, benzooxazolyl, and carbazolyl. Additionally, unless otherwise specified the heteroaryl group may be optionally substituted by various groups such as halo, hydroxy, alkyl, haloalkyl, alkoxy, nitro, cyano, alkylamine, carboxy or alkoxycarbonyl. Preferred heteroaryl group substituents include halo, alkyl, haloalkyl, hydroxy and alkoxy.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example, within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about”.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

When the methods include administering to a patient more than one active agent, the agents may be administered within 7, 6, 5, 4, 3, 2 or 1 days; within 24, 12, 6, 5, 4, 3, 2 or 1 hours, within 60, 50, 40, 30, 20, 10, 5 or 1 minutes; or substantially simultaneously. The methods, compositions and uses of the disclosure may include administering one or more agents to the patient by oral, systemic, parenteral, topical, intravenous, inhalational, or intramuscular administration.

Active ingredients or agents useful in the treatments of the disclosure include those described herein in any of their pharmaceutically acceptable forms, including isomers, salts, solvates, and polymorphs thereof, as well as racemic mixtures and prodrugs.

According to aspects illustrated herein, a treatment regimen of the present disclosure is suitable to prevent or reduce symptoms associated with obesity and/or diabetes.

Obesity is a condition that affects millions of in the United States. Individuals with a BMI of 25 to 29.9 are considered overweight, while individuals with a BMI of 30 or more are considered obese. Obesity or overweight may substantially increase the risk of morbidity from hypertension; dyslipidemia; type 2 diabetes and other disorders. Higher body weights are also associated with increases in all-cause mortality.

Diabetes mellitus is a serious metabolic disease that is defined by the presence of chronically elevated levels of blood glucose (hyperglycemia). This state of hyperglycemia is the result of a relative or absolute lack of activity of the peptide hormone, insulin. Insulin is produced and secreted by the B cells of the pancreas.

The term diabetes mellitus encompasses several different hyperglycemic states. These states include Type I (insulin-dependent diabetes mellitus or IDDM) and Type II (non-insulin dependent diabetes mellitus or NIDDM) diabetes.

In some embodiments, the pharmaceutical composition as described herein comprises an effective amount of a desired compound, such as opaganib. In some embodiments, the pharmaceutical composition is administered to a subject having a metabolic disease, such as obesity or type 2 diabetes.

In some embodiments, the compound, such as opaganib, is administered orally as a solid dosage form. In some embodiments, opaganib is administered orally one, two or more times per day.

Solid forms for oral administration may contain pharmaceutically acceptable binders, sweeteners, disintegrating agents, diluents, flavorings, coating agents, preservatives, lubricants, and/or time delay agents. Suitable binders include gum acacia, gelatin, corn starch, gum tragacanth, sodium alginate, carboxymethylcellulose or polyethylene glycol (PEG). Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavoring agents include peppermint oil, oil of wintergreen, cherry, orange, or raspberry flavoring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The compositions, uses and methods of the disclosure can include formulation(s) of compound(s) that, upon administration to a subject, result in a concentration of the compound(s) that prevent or treat symptoms associate with obesity and/or diabetes. The compound(s) may be contained in any appropriate amount in any suitable carrier substance, and are generally present in an amount of 1-95% by weight of the total weight of the composition. The composition may be provided in a dosage form that is suitable for the oral, parenteral (e.g., intravenously or intramuscularly), rectal, dermatological, cutaneous, nasal, vaginal, inhalant, skin (patch), ocular, intrathecal, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice.

Pharmaceutical compositions according to the disclosure or used in the uses and disclosure the disclosure may be formulated to release the active compound immediately upon administration or at any predetermined time or time period after administration. The latter types of compositions are generally known as controlled release formulations, which include (i) formulations that create substantially constant concentrations of the agent(s) of the disclosure within the body over an extended period of time; (ii) formulations that after a predetermined lag time create substantially constant concentrations of the agent(s) of the disclosure within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the combination to a particular target cell type.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound(s) are formulated with appropriate excipients into a pharmaceutical composition that, upon administration, releases the compound(s) in a controlled manner. Examples include single or multiple unit tablet or capsule compositions, oil solutions, suspensions, emulsions, microcapsules, molecular complexes, microspheres, nanoparticles, patches, and liposomes.

The dosage of a compound or a combination of compounds depends on several factors, including but not limited to the administration method, and the age, weight, and health of the patient to be treated.

As described above, the compound(s) in question may be administered orally in the form of tablets, capsules, elixirs or syrups, or rectally in the form of suppositories.

In some embodiments, the pharmaceutical composition is administered to a subject that is obese or overweight. In some embodiments, the pharmaceutical composition is administered to a subject that has diabetes. In some embodiments, the pharmaceutical composition is administered to a subject that has type II diabetes.

In some embodiments, an effective amount of the compound (such as opaganib) is from about 250 mg to about 500 mg, for example from about 250 mg to about 300 mg, from about 300 mg to about 350 mg, from about 350 mg to about 400 mg, from about 400 mg to about 450 mg, from about 450 mg to about 500 mg, about 250 mg, about 300 mg, about 350 mg, about 400 mg, about 450 mg, about 500 mg, or more.

In some embodiments, an effective amount of the compound (such as opaganib) is between about 15.0 mg/kg/day to about 20 mg/kg/day. In some embodiments, the compound (such as opaganib) is administered at a daily dosage ranging from about 2.5 mg/kg to about 22.5 mg/kg. In some embodiments, the compound (such as opaganib) is administered at a daily dosage ranging from about 3.5 mg/kg to about 21.5 mg/kg. In some embodiments, the compound (such as opaganib) is administered at a daily dosage ranging from about 4.5 mg/kg to about 20.5 mg/kg. In some embodiments, the compound (such as opaganib) is administered at a daily dosage ranging from about 5.5 mg/kg to about 19.5 mg/kg. In some embodiments, the compound (such as opaganib) is administered at a daily dosage ranging from about 6.5 mg/kg to about 18.5 mg/kg. In some embodiments, the compound (such as opaganib) is administered at a daily dosage ranging from about 7.5 mg/kg to about 17.5 mg/kg. In some embodiments, the compound (such as opaganib) is administered at a daily dosage ranging from about 8.5 mg/kg to about 16.5 mg/kg. In some embodiments, the compound (such as opaganib) is administered at a daily dosage ranging from about 9.5 mg/kg to about 15.5 mg/kg. In some embodiments, the compound (such as opaganib) is administered at a daily dosage ranging from about 10.5 mg/kg to about 14.5 mg/kg. In some embodiments, the compound (such as opaganib) is administered at a daily dosage ranging from about 11.5 mg/kg to about 13.5 mg/kg.

In some embodiments, an effective amount of the compound (such as opaganib) is administered once a day or twice a day or more. In some embodiments, an effective amount of the compound (such as opaganib) is administered twice a day. In some embodiments, the effective amount of the compound (such as opaganib) is administered for one or more days.

While various embodiments of the disclosure are described with respect to opaganib (i.e., 3-(4-chlorophenyl)-N-(pyridin-4-ylmethyl)-1-adamantanecarboxamide itself), a variety of compounds can be used. For example, in various embodiments of the methods, compositions and uses as described herein, the compound is 3-(4-chlorophenyl)-N-(pyridin-4-ylmethyl)-1-adamantanecarboxamide or a pharmaceutically acceptable salt or solvate (e.g., hydrate) thereof. The term “pharmaceutically acceptable salt or solvate” encompasses not only salts of the compound and solvates of the compound such as hydrates, but also solvates of salts of the compound. In various embodiments of the methods, compositions and uses as described herein, the compound is a compound as described in any of International Patent Application Publication no. 2006138660 and U.S. Pat. Nos. 8,063,248, 8,557,800, and RE49811, each of which is hereby incorporated herein by reference in its entirety, especially for their teachings of compounds, syntheses and pharmaceutical compositions. In various embodiments, the compound is a compound as described in any of International Patent Application Publication no. 2010105183 and U.S. Pat. No. 8,685,936, each of which is hereby incorporated herein by reference in its entirety, especially for their teachings of compounds, syntheses and pharmaceutical compositions. Any compound or group of compounds described in these publications can in various embodiments be suitable for use in the methods, compositions and uses of this disclosure.

For example, in various embodiments of the methods, compositions and uses as otherwise described herein, the compound is a compound of the formula

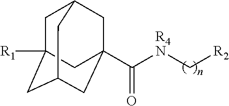

or a pharmaceutically acceptable salt or solvate (e.g., hydrate) thereof, wherein:
R₁ is phenyl, 4-chlorophenyl or 4-fluorophenyl;
R₂ is 4-pyridyl, optionally substituted with up to 4 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆) alkyl, and wherein each alkyl portion of a substituent is optionally further substituted with 1, 2, or 3 groups independently selected from halogen, CN, OH, and NH₂;
R₄ is H or alkyl; and
n is 1 or 2.
   In various such embodiments, R₁ is 4-fluorophenyl or 4-chlorophenyl.

In various embodiments of the methods, compositions and uses as otherwise described herein, the compound is:
3-(4-fluorophenyl)-N-(pyridin-4-ylmethyl)adamantane-1-carboxamide;
3-(4-chlorophenyl)-N-(pyridin-4-ylmethyl)adamantane-1-carboxamide; or
3-(4-chlorophenyl)-N-(2-pyridin-4-ylethyl)adamantane-1-carboxamide,
   or a pharmaceutically acceptable salt or solvate (e.g., hydrate) thereof.

In various embodiments of the methods, compositions and uses as otherwise described herein, the compound is a compound of the formula

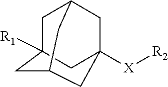

or a pharmaceutically acceptable salt or solvate (e.g., hydrate) thereof, wherein:
X is —C(R₃,R₄)N(R₅)— or —C(O)N(R₄)—;
R₁ is phenyl substituted with 1 to 5 groups that are independently halogen, haloalkyl, CONR′R″, —OC(O)NR′R″, —NR′C(O)R″, —CF₃, —OCF₃, —CN, —CO₂H, —S-alkyl, —SOR′R″, SO₂R′, wherein R′ and R″ are independently H or (C₁-C₆)alkyl, and wherein each alkyl portion of a substituent is optionally further substituted with 1, 2, or 3 groups independently selected from halogen, CN, OH, and NH₂;
R₂ is aryl, -alkyl-aryl, heteroaryl, -alkyl-heteroaryl, heterocycloalkyl or -alkyl-heterocycloalkyl;
R₃ is H or -alkyl;
wherein the alkyl and ring portion of each of the above R₂ and R₃ groups is optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂; and
R₄ and R₅ are independently H or alkyl.

In various such embodiments, X is —C(O)N(R₄)—. In various such embodiments, R₄ is H.

In various such embodiments, X is —C(R₃,R₄)N(R₅)—. In various such embodiments, R₃ is H or alkyl and R₄ is H.

In various such embodiments, R₁ is aryl (e.g., phenyl) substituted with one or two halogen groups.

In various such embodiments, R₂ is aryl (e.g., phenyl) or -alkyl-aryl (e.g., benzyl or phenethyl).

In various such embodiments, R₂ is phenyl, benzyl, phenethyl, or 3-phenylpropyl, in which the alkyl and ring portion of are optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂.

In various such embodiments, R₂ is heterocycloalkyl or -alkyl-heterocycloalkyl. In various such embodiments, each heterocycloalkyl is independently selected from a piperidinyl, a piperazinyl, a pyrrolidinyl, and a morpholinyl.

In various such embodiments, R₂ is piperidinyl, piperazinyl, 2-(piperazin-1-yl)ethyl, 2-(pyrrolidin-2-yl)ethyl, 3-(pyrrolidin-1-yl)propyl, or 2-(morpholin-4-yl)ethyl, in which the alkyl and ring portions of the above R₂ groups is optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, —C₁-C₆ alkoxy, -hydroxyalkyl, —CN, —CO₂H, —SH, —S-alkyl, —SOR′R″, —SO₂R′, —NO₂, or NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂.

In various such embodiments, R₂ is heteroaryl or alkyl-heteroaryl. In various such embodiments, each heteroaryl is independently selected from pyridinyl (e.g., pyridin-4-yl or pyridin-3-yl), indoyl (e.g., 1H-indol-5-yl), tetrazolyl (e.g., 1H-tetrazol-5-yl), imidazolyl (e.g., 3H-imidazol-4-y), benzothiazolyl (e.g., benzothiazol-2-yl), carbazolyl (e.g., carbazol-3-yl), benzooxazolyl (e.g., benzooxazol-2-yl), purinyl (e.g., purin-6-yl) and thienyl (e.g., thien-2-yl).

In various such embodiments, R₂ is pyridin-4-ylmethyl, pyridin-4-ylethyl, pyridin-3-ylmethyl, 1H-indol-5-yl, 1H-tetrazol-5-yl, 3H-imidazol-4-ylmethyl, benzothiazol-2-yl, carbazol-3-yl, benzooxazol-2-yl, purin-6-yl, or thien-2-yl, wherein the alkyl and ring portions of the above R₂ groups are optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, or —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂.

Particular compounds include those of the tables below:

TABLE 1

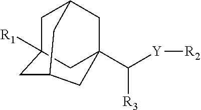

| Cmpd | Chemical name | Y | R₃ | R₁ | R₂ |
|---|---|---|---|---|---|
| 1 | 3-(4-Chloro-phenyl)-adamantane- 1-carboxylic acid isopropylamide | NH | ═O | 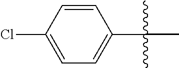 [SMILES: *c1ccc(Cl)cc1] | 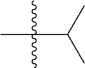 [SMILES: *C(C)C] |
| 2 | 3-(4-Chloro-phenyl)-adamantane- 1-carboxylic acid cyclopropylamide | NH | ═O | 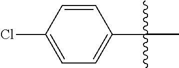 [SMILES: *c1ccc(Cl)cc1] | 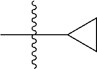 [SMILES: *C1CC1] |
| 3 | 3-(4-Chloro-phenyl)-adamantane- 1-carboxylic acid (2-ethylsulfanyl- ethyl)-amide | NH | ═O | 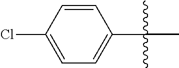 [SMILES: *c1ccc(Cl)cc1] | 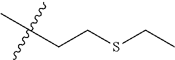 [SMILES: *CCSCC] |
| 4 | 3-(4-Chloro-phenyl)-adamantane- 1-carboxylic acid phenylamide | NH | ═O | 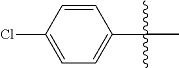 [SMILES: *c1ccc(Cl)cc1] | 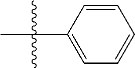 [SMILES: *c1ccccc1] |
| 5 | Adamantane-1-carboxylic acid (4- hydroxy-phenyl)-amide | NH | ═O | H | 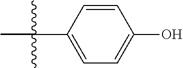 [SMILES: *c1ccc(O)cc1] |
| 6 | 3-(4-Chloro-phenyl)-adamantane- 1-carboxylic acid (4-hydroxy- phenyl)-amide | NH | ═O | 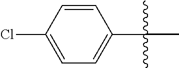 [SMILES: *c1ccc(Cl)cc1] | 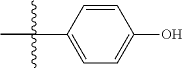 [SMILES: *c1ccc(O)cc1] |
| 7 | Acetic acid 4-{[3-(4-chloro- phenyl)-adamantane-1-carbonyl]- amino}-phenyl ester | NH | ═O | 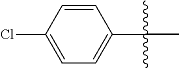 [SMILES: *c1ccc(Cl)cc1] | 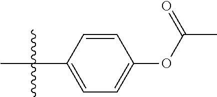 [SMILES: *c1ccc(OC(C)=O)cc1] |
| 8 | 3-(4-Chloro-phenyl)-adamantane- 1-carboxylic acid (2,4-dihydroxy- phenyl)-amide | NH | ═O | 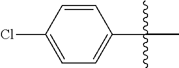 [SMILES: *c1ccc(Cl)cc1] | 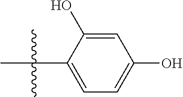 [SMILES: *c1ccc(O)cc1O] |
| 9 | 3-(4-Chloro-phenyl)-adamantane- 1-carboxylic acid (3- hydroxymethyl-phenyl)-amide | NH | ═O | 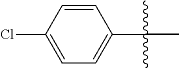 [SMILES: *c1ccc(Cl)cc1] | 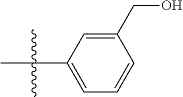 [SMILES: *c1cccc(CO)c1] |
| 10 | Adamantane-1-carboxylic acid (4- cyanomethyl-phenyl)-amide | NH | ═O | H | 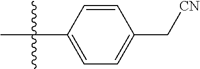 [SMILES: *c1ccc(CC#N)cc1] |
| 11 | 3-(4-Chloro-phenyl)-adamantane- 1-carboxylic acid (4-cyanomethyl- phenyl)-amide | NH | ═O | 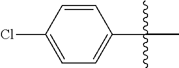 [SMILES: *c1ccc(Cl)cc1] | 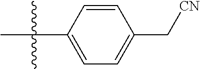 [SMILES: *c1ccc(CC#N)cc1] |
| 12 | 3-(4-Chloro-phenyl)-adamantane- 1-carboxylic acid benzylamide | NH | ═O | 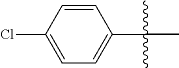 [SMILES: *c1ccc(Cl)cc1] | 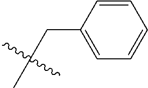 [SMILES: *Cc1ccccc1] |
| 13 | 3-(4-Chloro-phenyl)-adamantane- 1-carboxylic acid 4-tert-butyl- benzylamide | NH | ═O | 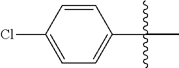 [SMILES: *c1ccc(Cl)cc1] | 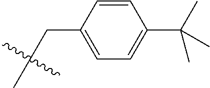 [SMILES: *Cc1ccc(C(C)(C)C)cc1] |
| 14 | 3-(4-Chloro-phenyl)-adamantane- 1-carboxylic acid 4- methylsulfanyl-benzylamide | NH | ═O | 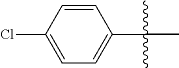 [SMILES: *c1ccc(Cl)cc1] | 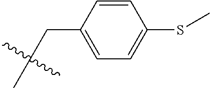 [SMILES: *Cc1ccc(SC)cc1] |
| 15 | 3-(4-Chloro-phenyl)-adamantane- 1-carboxylic acid 3- trifluoromethyl-benzylamide | NH | ═O | 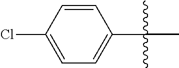 [SMILES: *c1ccc(Cl)cc1] | 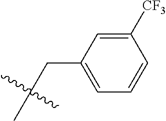 [SMILES: *Cc1cccc(C(F)(F)F)c1] |
| 16 | 3-(4-Chloro-phenyl)-adamantane- 1-carboxylic acid 4- trifluoromethyl-benzylamide | NH | ═O | 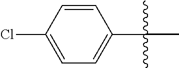 [SMILES: *c1ccc(Cl)cc1] | 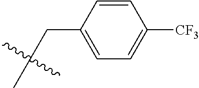 [SMILES: *Cc1ccc(C(F)(F)F)cc1] |
| 17 | 3-(4-Chloro-phenyl)-adamantane- 1-carboxylic acid 3,5-bis- trifluoromethyl-benzylamide | NH | ═O | 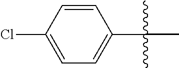 [SMILES: *c1ccc(Cl)cc1] | 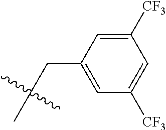 [SMILES: *Cc1cc(C(F)(F)F)cc(C(F)(F)F)c1] |
| 18 | 3-(4-Chloro-phenyl)-adamantane- 1-carboxylic acid 3-fluoro-5- trifluoromethyl-benzylamide | NH | ═O | 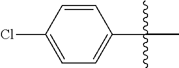 [SMILES: *c1ccc(Cl)cc1] | 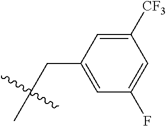 [SMILES: *Cc1cc(F)cc(C(F)(F)F)c1] |
| 19 | 3-(4-Chloro-phenyl)-adamantane- 1-carboxylic acid 2-fluoro-4- trifluoromethyl-benzylamide | NH | ═O | 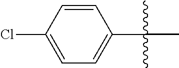 [SMILES: *c1ccc(Cl)cc1] | 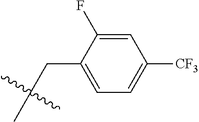 [SMILES: *Cc1ccc(C(F)(F)F)cc1F] |
| 20 | 3-(4-Chloro-phenyl)-adamantane- 1-carboxylic acid 3,5-difluoro- benzylamide | NH | ═O | 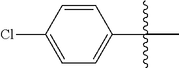 [SMILES: *c1ccc(Cl)cc1] | 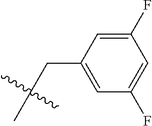 [SMILES: *Cc1cc(F)cc(F)c1] |
| 21 | 3-(4-Chloro-phenyl)-adamantane- 1-carboxylic acid 3,4-difluoro- benzylamide | NH | ═O | 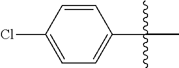 [SMILES: *c1ccc(Cl)cc1] | 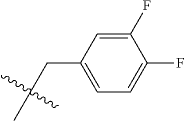 [SMILES: *Cc1ccc(F)c(F)c1] |
| 22 | 3-(4-Chloro-phenyl)-adamantane- 1-carboxylic acid 3,4,5-trifluoro- benzylamide | NH | ═O | 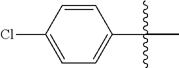 [SMILES: *c1ccc(Cl)cc1] | 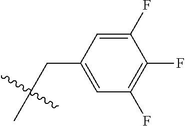 [SMILES: *Cc1cc(F)c(F)c(F)c1] |
| 23 | 3-(4-Chloro-phenyl)-adamantane- 1-carboxylic acid 3-chloro-4- fluoro-benzylamide | NH | ═O | 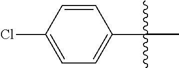 [SMILES: *c1ccc(Cl)cc1] | 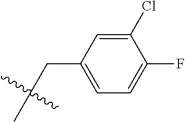 [SMILES: *Cc1ccc(F)c(Cl)c1] |
| 24 | 3-(4-Chloro-phenyl)-adamantane- 1-carboxylic acid 4-fluoro-3- trifluoromethyl-benzylamide | NH | ═O | 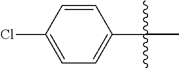 [SMILES: *c1ccc(Cl)cc1] | 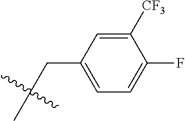 [SMILES: *Cc1ccc(F)c(C(F)(F)F)c1] |
| 25 | 3-(4-Chloro-phenyl)-adamantane- 1-carboxylic acid 2-chloro-4- fluoro-benzylamide | NH | ═O | 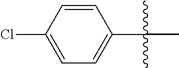 [SMILES: *c1ccc(Cl)cc1] | 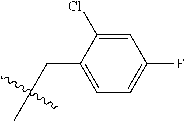 [SMILES: *Cc1ccc(F)cc1Cl] |
| 26 | 3-(4-Chloro-phenyl)-adamantane- 1-carboxylic acid 4-chloro-3- trifluoromethyl-benzylamide | NH | ═O | 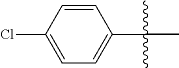 [SMILES: *c1ccc(Cl)cc1] | 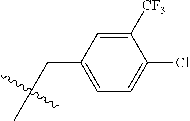 [SMILES: *Cc1ccc(Cl)c(C(F)(F)F)c1] |
| 27 | 3-(4-Chloro-phenyl)-adamantane- 1-carboxylic acid 3-aminomethyl- 2,4,5,6-tetrachloro-benzylamide | NH | ═O | 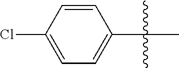 [SMILES: *c1ccc(Cl)cc1] | 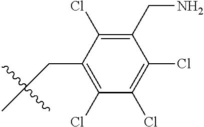 [SMILES: *Cc1c(Cl)c(Cl)c(Cl)c(CN)c1Cl] |
| 28 | 3-(4-Chloro-phenyl)-adamantane- 1-carboxylic acid [1-(4-chloro- phenyl)-ethyl]-amide | NH | ═O | 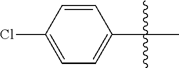 [SMILES: *c1ccc(Cl)cc1] | 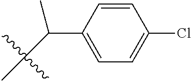 [SMILES: *C(C)c1ccc(Cl)cc1] |
| 29 | 3-(4-Chloro-phenyl)-adamantane- 1-carboxylic acid [1-(4-bromo- phenyl)-ethyl]-amide | NH | ═O | 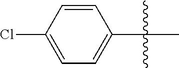 [SMILES: *c1ccc(Cl)cc1] | 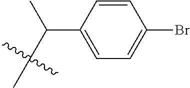 [SMILES: *C(C)c1ccc(Br)cc1] |
| 30 | 3-(4-Chloro-phenyl)-adamantane- 1-carboxylic acid 4- methanesulfonyl-benzylamide | NH | ═O | 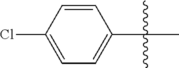 [SMILES: *c1ccc(Cl)cc1] | 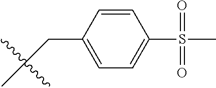 [SMILES: *Cc1ccc(S(C)(=O)=O)cc1] |
| 31 | 3-(4-Chloro-phenyl)-adamantane- 1-carboxylic acid 4- dimethylamino-benzylamide | NH | ═O | 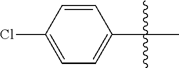 [SMILES: *c1ccc(Cl)cc1] | 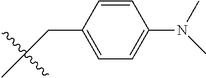 [SMILES: *Cc1ccc(N(C)C)cc1] |
| 32 | 3-(4-Chloro-phenyl)-adamantane- 1-carboxylic acid 4- trifluoromethoxy-benzylamide | NH | ═O | 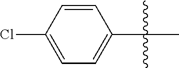 [SMILES: *c1ccc(Cl)cc1] | 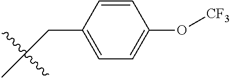 [SMILES: *Cc1ccc(OC(F)(F)F)cc1] |
| 33 | 3-(4-Chloro-phenyl)-adamantane- 1-carboxylic acid 3- trifluoromethoxy-benzylamide | NH | ═O | 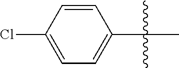 [SMILES: *c1ccc(Cl)cc1] | 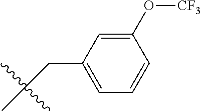 [SMILES: *Cc1cccc(OC(F)(F)F)c1] |
| 34 | 3-(4-Chloro-phenyl)-adamantane- 1-carboxylic acid 4-phenoxy- benzylamide | NH | ═O | 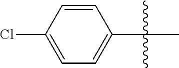 [SMILES: *c1ccc(Cl)cc1] | 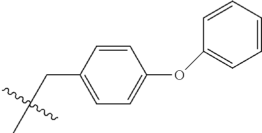 [SMILES: *Cc1ccc(Oc2ccccc2)cc1] |
| 35 | Adamantane-1-carboxylic acid 3,4- dihydroxy-benzylamide | NH | ═O | H | 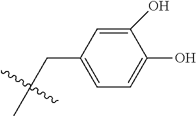 [SMILES: *Cc1ccc(O)c(O)c1] |
| 36 | 3-(4-Chloro-phenyl)-adamantane- 1-carboxylic acid 3,4-dihydroxy- benzylamide | NH | ═O | 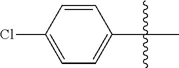 [SMILES: *c1ccc(Cl)cc1] | 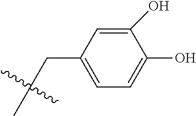 [SMILES: *Cc1ccc(O)c(O)c1] |
| 37 | 3-(4-Chloro-phenyl)-adamantane- 1-carboxylic acid phenethyl-amide | NH | ═O | 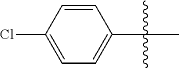 [SMILES: *c1ccc(Cl)cc1] | 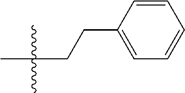 [SMILES: *CCc1ccccc1] |
| 38 | 3-(4-Chloro-phenyl)-adamantane- 1-carboxylic acid [2-(4-fluoro- phenyl)-ethyl]-amide | NH | ═O | 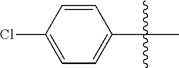 [SMILES: *c1ccc(Cl)cc1] | 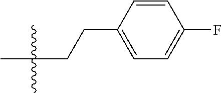 [SMILES: *CCc1ccc(F)cc1] |
| 39 | 3-(4-Chloro-phenyl)-adamantane- 1-carboxylic acid [2-(4-bromo- phenyl)-ethyl]-amide | NH | ═O | 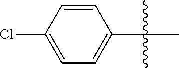 [SMILES: *c1ccc(Cl)cc1] | 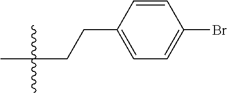 [SMILES: *CCc1ccc(Br)cc1] |
| 40 | 3-(4-Chloro-phenyl)-adamantane- 1-carboxylic acid [2-(4-hydroxy- phenyl)-ethyl]-amide | NH | ═O | 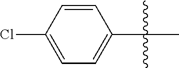 [SMILES: *c1ccc(Cl)cc1] | 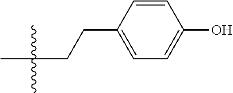 [SMILES: *CCc1ccc(O)cc1] |
| 41 | 3-(4-Chloro-phenyl)-adamantane- 1-carboxylic acid 4-phenoxy- benzylamide | NH | ═O | 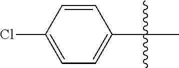 [SMILES: *c1ccc(Cl)cc1] | 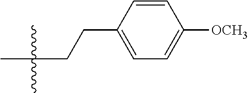 [SMILES: *CCc1ccc(OC)cc1] |
| 42 | 3-(4-Chloro-phenyl)-adamantane- 1-carboxylic acid [2-(3-bromo-4- methoxy-phenyl)-ethyl]-amide | NH | ═O | 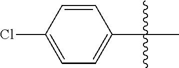 [SMILES: *c1ccc(Cl)cc1] | 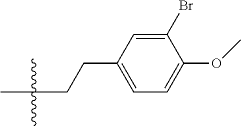 [SMILES: *CCc1ccc(OC)c(Br)c1] |
| 43 | Adamantane-1-carboxylic acid [2- (3,4-dihydroxy-phenyl)-ethyl]- amide | NH | ═O | H | 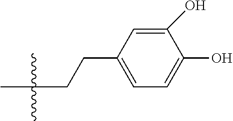 [SMILES: *CCc1ccc(O)c(O)c1] |
| 44 | 3-(4-Chloro-phenyl)-adamantane- 1-carboxylic acid [2-(3,4- dihydroxy-phenyl)-ethyl]-amide | NH | ═O | 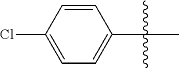 [SMILES: *c1ccc(Cl)cc1] | 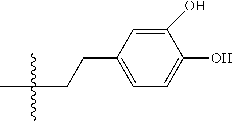 [SMILES: *CCc1ccc(O)c(O)c1] |
| 45 | 3-(4-Chloro-phenyl)-adamantane- 1-carboxylic acid (2- benzo[1,3]dioxol-5-yl-ethyl)-amide | NH | ═O | 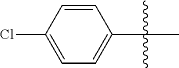 [SMILES: *c1ccc(Cl)cc1] | 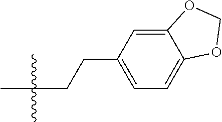 [SMILES: *CCc1ccc2c(c1)OCO2] |
| 46 | 3-(4-Chloro-phenyl)-adamantane- 1-carboxylic acid [2-(3-phenoxy- phenyl)-ethyl]-amide | NH | ═O | 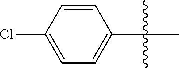 [SMILES: *c1ccc(Cl)cc1] | 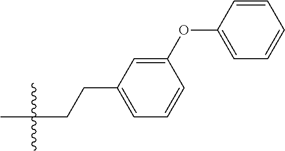 [SMILES: *CCc1cccc(Oc2ccccc2)c1] |
| 47 | 3-(4-Chloro-phenyl)-adamantane- 1-carboxylic acid [2-(4-phenoxy- phenyl)-ethyl]-amide | NH | ═O | 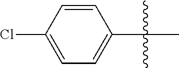 [SMILES: *c1ccc(Cl)cc1] | 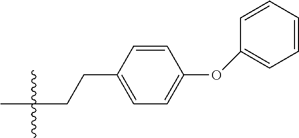 [SMILES: *CCc1ccc(Oc2ccccc2)cc1] |
| 48 | 3-(4-Chloro-phenyl)-adamantane- 1-carboxylic acid (3-phenyl- propyl)-amide | NH | ═O | 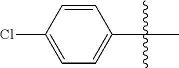 [SMILES: *c1ccc(Cl)cc1] | 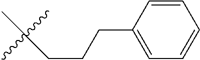 [SMILES: *CCCc1ccccc1] |
| 49 | 3-(4-Chloro-phenyl)-adamantane- 1-carboxylic acid (biphenyl-4- ylmethyl)-amide | NH | ═O | 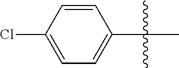 [SMILES: *c1ccc(Cl)cc1] | 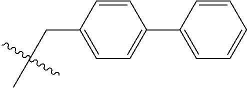 [SMILES: *Cc1ccc(-c2ccccc2)cc1] |
| 50 | Adamantane-1-carboxylic acid (1- methyl-piperidin-4-yl)-amide | NH | ═O | H | 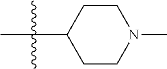 [SMILES: *C1CCN(C)CC1] |
| 51 | 3-(4-Chloro-phenyl)-adamantane- 1-carboxylic acid (1-methyl- piperidin-4-yl)-amide | NH | ═O | 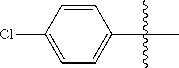 [SMILES: *c1ccc(Cl)cc1] | 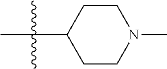 [SMILES: *C1CCN(C)CC1] |
| 52 | 3-(4-Chloro-phenyl)-adamantane- 1-carboxylic acid (4-methyl- piperazin-1-yl)-amide | NH | ═O | 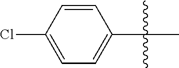 [SMILES: *c1ccc(Cl)cc1] | 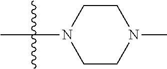 [SMILES: *N1CCN(C)CC1] |
| 53 | 3-(4-Chloro-phenyl)-adamantane- 1-carboxylic acid (3-tert- butylamino-propyl)-amide | NH | ═O | 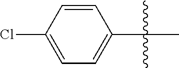 [SMILES: *c1ccc(Cl)cc1] | 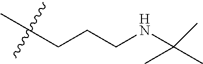 [SMILES: *CCCNC(C)(C)C] |
| 54 | 3-(4-Chloro-phenyl)-adamantane- 1-carboxylic acid (3-pyrrolidin-1- yl-propyl)-amide | NH | ═O | 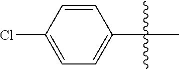 [SMILES: *c1ccc(Cl)cc1] | 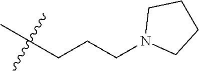 [SMILES: *CCCN1CCCC1] |
| 55 | 3-(4-Chloro-phenyl)-adamantane- 1-carboxylic acid [3-(2-oxo- pyrrolidin-1-yl)-propyl]-amide | NH | ═O | 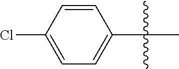 [SMILES: *c1ccc(Cl)cc1] | 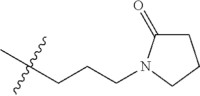 [SMILES: *CCCN1CCCC1=O] |
| 56 | Adamantane-1-carboxylic acid [2- (1-methyl-pyrrolidin-2-yl)-ethyl]- amide | NH | ═O | H | 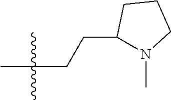 [SMILES: *CCC1CCCN1C] |
| 57 | 3-(4-Chloro-phenyl)-adamantane- 1-carboxylic acid [2-(1-methyl- pyrrolidin-2-yl)-ethyl]-amide | NH | ═O | 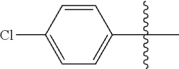 [SMILES: *c1ccc(Cl)cc1] | 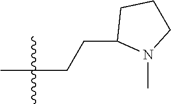 [SMILES: *CCC1CCCN1C] |
| 58 | 3-(4-Chloro-phenyl)-adamantane- 1-carboxylic acid (2-morpholin-4- yl-ethyl)-amide | NH | ═O | 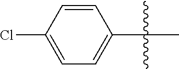 [SMILES: *c1ccc(Cl)cc1] | 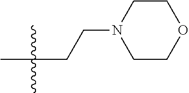 [SMILES: *CCN1CCOCC1] |
| 59 | 3-(4-Chloro-phenyl)-adamantane- 1-carboxylic acid (2-piperazin-1- yl-ethyl)-amide | NH | ═O | 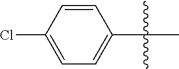 [SMILES: *c1ccc(Cl)cc1] | 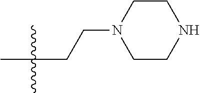 [SMILES: *CCN1CCNCC1] |
| 60 | Adamantane-1-carboxylic acid (pyridin-4-ylmethyl)-amide | NH | ═O | H | 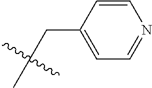 [SMILES: *Cc1ccncc1] |
| 61 | 3-(4-Fluoro-phenyl)-adamantane-1- carboxylic acid (pyridin-4- ylmethyl)-amide | NH | ═O | 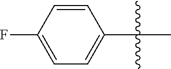 [SMILES: *c1ccc(F)cc1] | 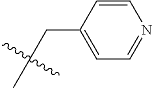 [SMILES: *Cc1ccncc1] |
| 62 | 3-(4-Chloro-phenyl)-adamantane- 1-carboxylic acid (pyridin-4- ylmethyl)-amide | NH | ═O | 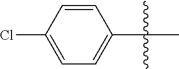 [SMILES: *c1ccc(Cl)cc1] | 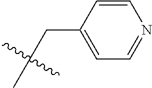 [SMILES: *Cc1ccncc1] |
| 63 | Adamantane-1-carboxylic acid (pyridin-4-ylmethyl)-amide | NH | ═O | H | 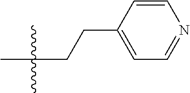 [SMILES: *CCc1ccncc1] |
| 64 | 3-(4-Chloro-phenyl)-adamantane- 1-carboxylic acid (2-pyridin-4-yl- ethyl)-amide | NH | ═O | 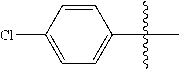 [SMILES: *c1ccc(Cl)cc1] | 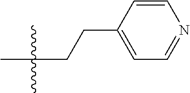 [SMILES: *CCc1ccncc1] |
| 65 | 3-(4-Chloro-phenyl)-adamantane- 1-carboxylic acid (3-imidazol-1-yl- propyl)-amide | NH | ═O | 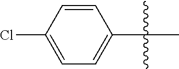 [SMILES: *c1ccc(Cl)cc1] | 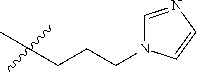 [SMILES: *CCCn1ccnc1] |
| 66 | 3-(4-Chloro-phenyl)-adamantane- 1-carboxylic acid (2-methyl-1H- indol-5-yl)-amide | NH | ═O | 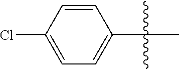 [SMILES: *c1ccc(Cl)cc1] | 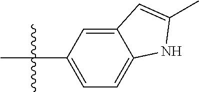 [SMILES: *c1ccc2[nH]c(C)cc2c1] |
| 67 | 3-(4-Chloro-phenyl)-adamantane- 1-carboxylic acid (1H-tetrazol-5- yl)-amide | NH | ═O | 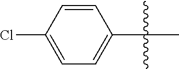 [SMILES: *c1ccc(Cl)cc1] | 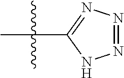 [SMILES: *c1nnn[nH]1] |
| 68 | 3-(4-Chloro-phenyl)-adamantane- 1-carboxylic acid (9-ethyl-9H- carbazol-3-yl)-amide | NH | ═O | 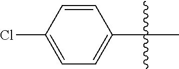 [SMILES: *c1ccc(Cl)cc1] | 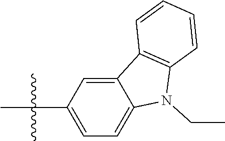 [SMILES: *c1ccc2c(c1)c1ccccc1n2CC] |
| 69 | Adamantane-1-carboxylic acid [4- (4-chloro-phenyl)-thiazol-2-yl]- amide | NH | ═O | H | 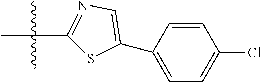 [SMILES: *c1ncc(-c2ccc(Cl)cc2)s1] |
| 70 | 3-(4-Chloro-phenyl)-adamantane- 1-carboxylic acid [4-(4-chloro- phenyl)-thiazol-2-yl]-amide | NH | ═O | 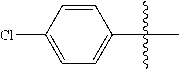 [SMILES: *c1ccc(Cl)cc1] | 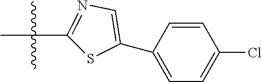 [SMILES: *c1ncc(-c2ccc(Cl)cc2)s1] |
| 71 | 3-(4-Chloro-phenyl)-adamantane- 1-carboxylic acid benzothiazol-2- ylamide | NH | ═O | 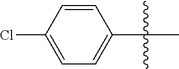 [SMILES: *c1ccc(Cl)cc1] | 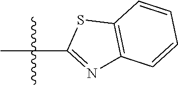 [SMILES: *c1nc2ccccc2s1] |
| 72 | 3-(4-Chloro-phenyl)-adamantane- 1-carboxylic acid (5-chloro- benzooxazol-2-yl)-amide | NH | ═O | 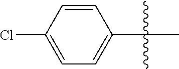 [SMILES: *c1ccc(Cl)cc1] | 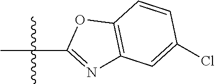 [SMILES: *c1nc2cc(Cl)ccc2o1] |
| 73 | 3-(4-Chloro-phenyl)-adamantane- 1-carboxylic acid (9H-purin-6-yl)- amide | NH | ═O | 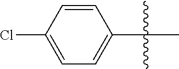 [SMILES: *c1ccc(Cl)cc1] | 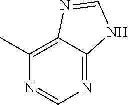 |
| 75 | [3-(4-Chloro-phenyl)-adamantan-1- ylmethyl]-isopropyl-amine | NH | H | 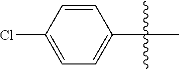 [SMILES: *c1ccc(Cl)cc1] | 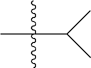 [SMILES: *C(C)C] |
| 76 | 4- and -phenol | NH | H | 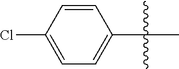 [SMILES: *c1ccc(Cl)cc1] | 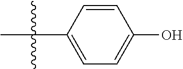 [SMILES: *c1ccc(O)cc1] |
| 77 | [3-(4-Chloro-phenyl)-adamantan-1- ylmethyl]-(4-trifluoromethyl- benzyl)-amine | NH | H | 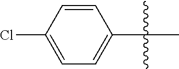 [SMILES: *c1ccc(Cl)cc1] | 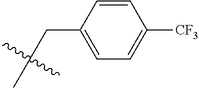 [SMILES: *Cc1ccc(C(F)(F)F)cc1] |
| 78 | [3-(4-Chloro-phenyl)-adamantan-1- ylmethyl]-(2-fluoro-4- trifluoromethyl-benzyl)-amine | NH | H | 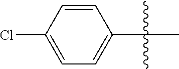 [SMILES: *c1ccc(Cl)cc1] | 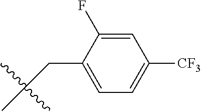 [SMILES: *Cc1ccc(C(F)(F)F)cc1F] |
| 79 | [3-(4-Chloro-phenyl)-adamantan-1- ylmethyl]-(4-fluoro-3- trifluoromethyl-benzyl)-amine | NH | H | 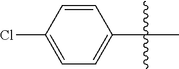 [SMILES: *c1ccc(Cl)cc1] | 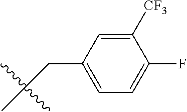 [SMILES: *Cc1ccc(F)c(C(F)(F)F)c1] |
| 80 | [3-(4-Chloro-phenyl)-adamantan-1- ylmethyl]-(4-trifluoromethoxy- benzyl)-amine | NH | H | 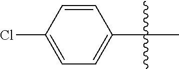 [SMILES: *c1ccc(Cl)cc1] | 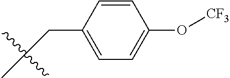 [SMILES: *Cc1ccc(OC(F)(F)F)cc1] |
| 81 | [3-(4-Chloro-phenyl)-adamantan-1- ylmethyl]-[2-(3-phenoxy-phenyl)- ethyl]-amine | NH | H | 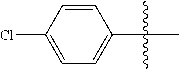 [SMILES: *c1ccc(Cl)cc1] | 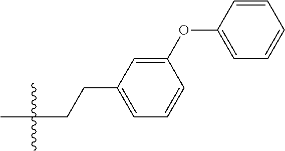 [SMILES: *CCc1cccc(Oc2ccccc2)c1] |
| 82 | [3-(4-Chloro-phenyl)-adamantan-1- ylmethyl]-(1-methyl-piperidin-4- yl)-amine | NH | H | 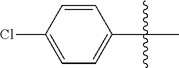 [SMILES: *c1ccc(Cl)cc1] | 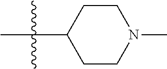 [SMILES: *C1CCN(C)CC1] |
| 83 | [3-(4-Chloro-phenyl)-adamantan-1- ylmethyl]-(4-methyl-piperazin-1- yl)-amine | NH | H | 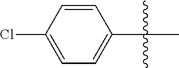 [SMILES: *c1ccc(Cl)cc1] | 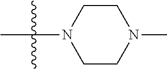 [SMILES: *N1CCN(C)CC1] |
| 84 | N-tert-Butyl-N′-[3-(4-chloro- phenyl)-adamantan-1-ylmethyl]- propane-1,3-diamine | NH | H | 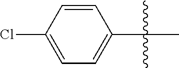 [SMILES: *c1ccc(Cl)cc1] | 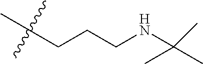 [SMILES: *CCCNC(C)(C)C] |
| 85 | [3-(4-Chloro-phenyl)-adamantan-1- ylmethyl]-(3-pyrrolidin-1-yl- propyl)-amine | NH | H | 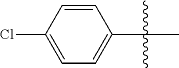 [SMILES: *c1ccc(Cl)cc1] | 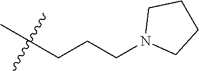 [SMILES: *CCCN1CCCC1] |
| 86 | [3-(4-Chloro-phenyl)-adamantan-1- ylmethyl]-[2-(1-methyl-pyrrolidin- 2-yl)-ethyl]-amine | NH | H | 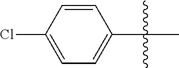 [SMILES: *c1ccc(Cl)cc1] | 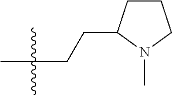 [SMILES: *CCC1CCCN1C] |
| 87 | [3-(4-Chloro-phenyl)-adamantan-1- ylmethyl]-(2-morpholin-4-yl- ethyl)-amine | NH | H | 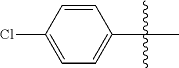 [SMILES: *c1ccc(Cl)cc1] | 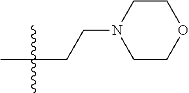 [SMILES: *CCN1CCOCC1] |
| 88 | [3-(4-Chloro-phenyl)-adamantan-1- ylmethyl]-pyridin-4-ylmethyl- amine | NH | H | 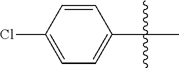 [SMILES: *c1ccc(Cl)cc1] | 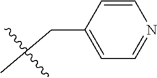 [SMILES: *Cc1ccncc1] |
| 89 | [3-(4-Chloro-phenyl)-adamantan-1- ylmethyl]-(9-ethyl-9H-carbazol-3- yl)-amine | NH | H | 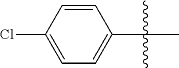 [SMILES: *c1ccc(Cl)cc1] | 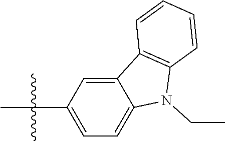 [SMILES: *c1ccc2c(c1)c1ccccc1n2CC] |
| 90 | [3-(4-Chloro-phenyl)-adamantan-1- ylmethyl]-[5-(4-chloro-phenyl)- thiazol-2-yl]-amine | NH | H | 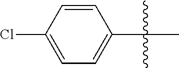 [SMILES: *c1ccc(Cl)cc1] | 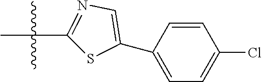 [SMILES: *c1ncc(-c2ccc(Cl)cc2)s1] |
| 91 | 1-[3-(4-Chloro-phenyl)-adamantan- 1-yl]-ethylamine | NH | CH₃ | 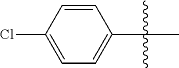 [SMILES: *c1ccc(Cl)cc1] | H |
| 92 | {1-[3-(4-Chloro-phenyl)- adamantan-1-yl]-ethyl}-isopropyl- amine | NH | CH₃ | 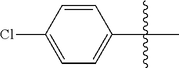 [SMILES: *c1ccc(Cl)cc1] | 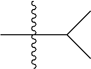 [SMILES: *C(C)C] |
| 93 | Phenyl-[1-(3-phenyl-adamantan-1- yl)-ethyl]-amine | NH | CH₃ | 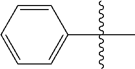 [SMILES: *c1ccccc1] | 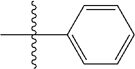 [SMILES: *c1ccccc1] |
| 94 | {1-[3-(4-Fluoro-phenyl)- adamantan-1-yl]-ethyl}-phenyl- amine | NH | CH₃ | 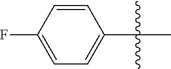 [SMILES: *c1ccc(F)cc1] | 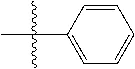 [SMILES: *c1ccccc1] |
| 95 | {1-[3-(4-Chloro-phenyl)- adamantan-1-yl]-ethyl}-phenyl- amine | NH | CH₃ | 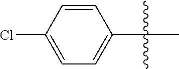 [SMILES: *c1ccc(Cl)cc1] | 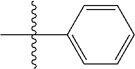 [SMILES: *c1ccccc1] |
| 96 | (1-Adamantan-1-yl-ethyl)-benzyl- amine | NH | CH₃ | H | 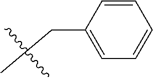 [SMILES: *Cc1ccccc1] |
| 97 | Benzyl-[1-(3-phenyl-adamantan-1- yl)-ethyl]-amine | NH | CH₃ | 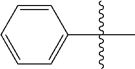 [SMILES: *c1ccccc1] | 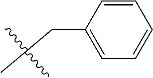 [SMILES: *Cc1ccccc1] |
| 98 | Benzyl-{1-[3-(4-fluoro-phenyl)- adamantan-1-yl]-ethyl}-amine | NH | CH₃ | 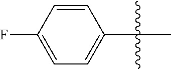 [SMILES: *c1ccc(F)cc1] | 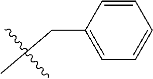 [SMILES: *Cc1ccccc1] |
| 99 | Benzyl-{1-[3-(4-chloro-phenyl)- adamantan-1-yl]-ethyl}-amine | NH | CH₃ | 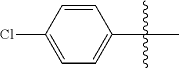 [SMILES: *c1ccc(Cl)cc1] | 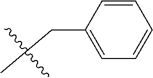 [SMILES: *Cc1ccccc1] |
| 100 | (4-tert-Butyl-benzyl)-{1-[3-(4- chloro-phenyl)-adamantan-1-yl]- ethyl}-amine | NH | CH₃ | 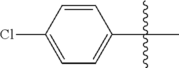 [SMILES: *c1ccc(Cl)cc1] | 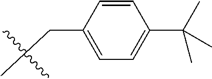 [SMILES: *Cc1ccc(C(C)(C)C)cc1] |
| 101 | [1-(4-Bromo-phenyl)-ethyl]-{1-[3- (4-chloro-phenyl)-adamantan-1- yl]-ethyl}-amine | NH | CH₃ | 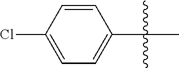 [SMILES: *c1ccc(Cl)cc1] | 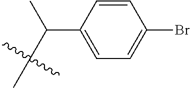 [SMILES: *C(C)c1ccc(Br)cc1] |
| 102 | (1-Adamantan-1-yl-ethyl)-[2-(4- bromo-phenyl)-ethyl]-amine | NH | CH₃ | H | 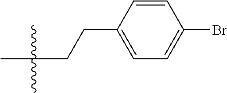 [SMILES: *CCc1ccc(Br)cc1] |
| 103 | [2-(4-Bromo-phenyl)-ethyl]-{1-[3- (4-chloro-phenyl)-adamantan-1- yl]-ethyl}-amine | NH | CH₃ | 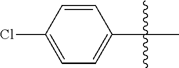 [SMILES: *c1ccc(Cl)cc1] | 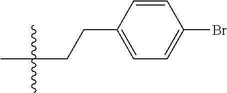 [SMILES: *CCc1ccc(Br)cc1] |
| 104 | (1-Adamantan-1-yl-ethyl)-(1- methyl-piperidin-4-yl)-amine | NH | CH₃ | H | 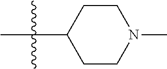 [SMILES: *C1CCN(C)CC1] |
| 105 | (1-Methyl-piperidin-4-yl)-[1-(3- phenyl-adamantan-1-yl)-ethyl]- amine | NH | CH₃ | 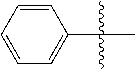 [SMILES: *c1ccccc1] | 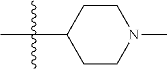 [SMILES: *C1CCN(C)CC1] |
| 106 | {1-[3-(4-Fluoro-phenyl)- adamantan-1-yl]-ethyl}-(1-methyl- piperidin-4-yl)-amine | NH | CH₃ | 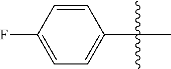 [SMILES: *c1ccc(F)cc1] | 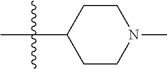 [SMILES: *C1CCN(C)CC1] |
| 107 | {1-[3-(4-Chloro-phenyl)- adamantan-1-yl]-ethyl}-(1-methyl- piperidin-4-yl)-amine | NH | CH₃ | 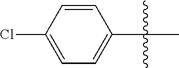 [SMILES: *c1ccc(Cl)cc1] | 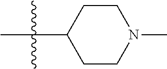 [SMILES: *C1CCN(C)CC1] |
| 108 | {1-[3-(4-Chloro-phenyl)- adamantan-1-yl]-ethyl}-(4-methyl- piperazin-1-yl)-amine | NH | CH₃ | 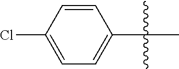 [SMILES: *c1ccc(Cl)cc1] | 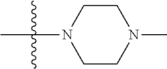 [SMILES: *N1CCN(C)CC1] |
| 109 | {1-[3-(Phenyl)-adamantan-1-yl]- ethyl}-pyridin-4-ylmethyl-amine | NH | CH₃ | 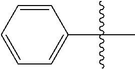 [SMILES: *c1ccccc1] | 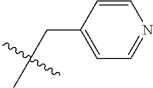 [SMILES: *Cc1ccncc1] |
| 110 | {1-[3-(4-Chloro-phenyl)- adamantan-1-yl]-ethyl}-(6-chloro- pyridin-3-ylmethyl)-amine | NH | CH₃ | 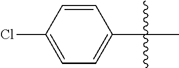 [SMILES: *c1ccc(Cl)cc1] | 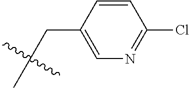 [SMILES: *Cc1ccc(Cl)nc1] |
| 111 | {1-[3-(4-Chloro-phenyl)- adamantan-1-yl]-ethyl}-(2-pyridin- 4-yl-ethyl)-amine | NH | CH₃ | 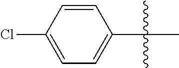 [SMILES: *c1ccc(Cl)cc1] | 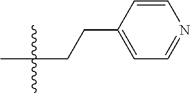 [SMILES: *CCc1ccncc1] |
| 112 | {1-[3-(4-Chloro-phenyl)- adamantan-1-yl]-ethyl}-(3H- imidazol-4-ylmethyl)-amine | NH | CH₃ | 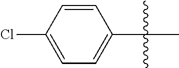 [SMILES: *c1ccc(Cl)cc1] | 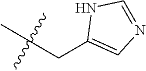 [SMILES: *Cc1cnc[nH]1] |
| 113 | {1-[3-(4-Chloro-phenyl)- adamantan-1-yl]-ethyl}-(2-methyl- 1H-indol-5-yl)-amine | NH | CH₃ | 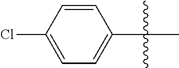 [SMILES: *c1ccc(Cl)cc1] | 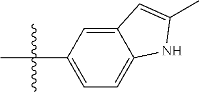 [SMILES: *c1ccc2[nH]c(C)cc2c1] |
| 114 | {1-[3-(4-Chloro-phenyl)- adamantan-1-yl]-ethyl}-(9-ethyl- 9H-carbazol-3-yl)-amine | NH | CH₃ | 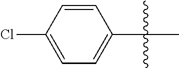 [SMILES: *c1ccc(Cl)cc1] | 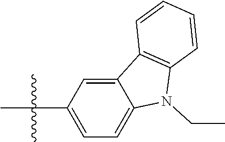 [SMILES: *c1ccc2c(c1)c1ccccc1n2CC] |
| 115 | {1-[3-(4-Chloro-phenyl)- adamantan-1-yl]-ethyl}-(9-ethyl- 9H-carbazol-3-ylmethyl)-amine | NH | CH₃ | 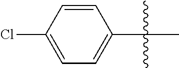 [SMILES: *c1ccc(Cl)cc1] | 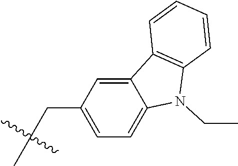 [SMILES: *Cc1ccc2c(c1)c1ccccc1n2CC] |
| 116 | 9-Ethyl-9H-carbazole-3-carboxylic acid {1-[3-(4-chloro-phenyl)- adamantan-1-yl]-ethyl}-amide | NH | CH₃ | 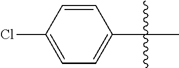 [SMILES: *c1ccc(Cl)cc1] | 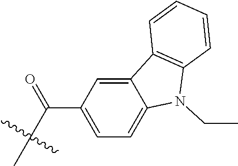 [SMILES: *C(=O)c1ccc2c(c1)c1ccccc1n2CC] |
| 117 | 1-{1-[3-(4-Chloro-phenyl)- adamantan-1-yl]-ethyl}-3-(4- chloro-3-trifluoromethyl-phenyl)- urea | NH | CH₃ | 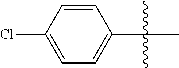 [SMILES: *c1ccc(Cl)cc1] | 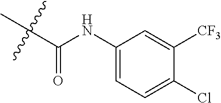 [SMILES: *C(=O)Nc1ccc(Cl)c(C(F)(F)F)c1] |
| 118 | 1-{1-[3-(4-Chloro-phenyl)- adamantan-1-yl]-ethyl}-3-(4- chloro-3-trifluoromethyl-phenyl)- urea | NH | CH₃ | 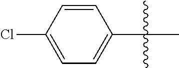 [SMILES: *c1ccc(Cl)cc1] | 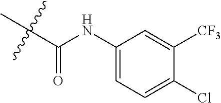 [SMILES: *C(=O)Nc1ccc(Cl)c(C(F)(F)F)c1] |
| 119 | (4-Bromo-thiophen-2-ylmethyl)- {1-[3-(4-chloro-phenyl)- adamantan-1-yl]-ethyl}-amine | NH | CH₃ | 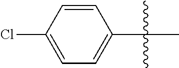 [SMILES: *c1ccc(Cl)cc1] | 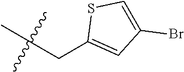 [SMILES: *Cc1cc(Br)cs1] |
| 120 | {1-[3-(4-Chloro-phenyl)- adamantan-1-yl]-ethyl}-(4-phenyl- thiophen-2-ylmethyl)-amine | NH | CH₃ | 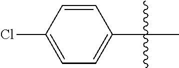 [SMILES: *c1ccc(Cl)cc1] | 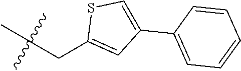 [SMILES: *Cc1cc(-c2ccccc2)cs1] |

TABLE 2
Representative compounds of the invention.

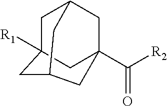

| Cmpd | Chemical name | R₁ | R₂ |
|---|---|---|---|
| 121 | 3-Phenyl-adamantane- 1-carboxylic acid | 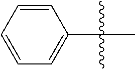 [SMILES: *c1ccccc1] | OH |
| 122 | 3-(4-Fluoro-phenyl)- adamantane-1-carboxylic acid | 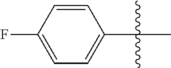 [SMILES: *c1ccc(F)cc1] | OH |
| 123 | 3-(4-Chloro-phenyl)- adamantane-1-carboxylic acid | 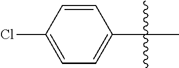 [SMILES: *c1ccc(Cl)cc1] | OH |
| 124 | 1-Adamantan-1-yl-ethanone | H | CH₃ |
| 125 | 1-(3-Phenyl-adamantan- 1-yl)-ethanone | 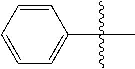 [SMILES: *c1ccccc1] | CH₃ |
| 126 | 1-[3-(4-Fluoro-phenyl)- adamantan-1-yl]-ethanone | 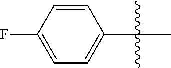 [SMILES: *c1ccc(F)cc1] | CH₃ |
| 127 | 1-[3-(4-Chloro-phenyl)- adamantan-1-yl]-ethanone | 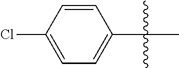 [SMILES: *c1ccc(Cl)cc1] | CH₃ |
| 128 | 2-(Adamantane-1- carbonyl)-malonic acid dimethyl ester | H | 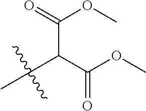 [SMILES: *C(C(=O)OC)C(=O)OC] |
| 129 | 2-[3-(4-Chloro-phenyl)- adamantane-1-carbonyl]- malonic acid dimethyl ester | 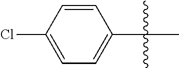 [SMILES: *c1ccc(Cl)cc1] | 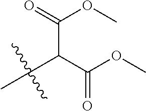 [SMILES: *C(C(=O)OC)C(=O)OC] |
| 130 | 3-(4-Chloro-phenyl)- 1-[3-(4-chloro-phenyl)- adamantan-1-yl]-propenone | 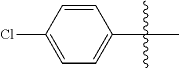 [SMILES: *c1ccc(Cl)cc1] | 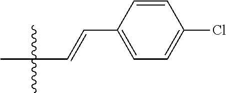 [SMILES: */C=C/c1ccc(Cl)cc1] |
| 131 | 4-{3-[3-(4-Chloro-phenyl)- adamantan-1-yl]-3-oxo- propenyl}-benzonitrile | 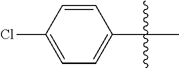 [SMILES: *c1ccc(Cl)cc1] | 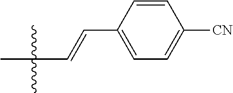 [SMILES: */C=C/c1ccc(C#N)cc1] |
| 132 | 1-[3-(4-Chloro-phenyl)- adamantan-1-yl]-3-(4- hydroxy-phenyl)-propenone | 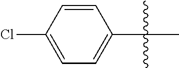 [SMILES: *c1ccc(Cl)cc1] | 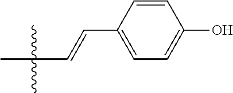 [SMILES: */C=C/c1ccc(O)cc1] |
| 133 | 1-[3-(4-Chloro-phenyl)- adamantan-1-yl]-3- naphthalen-2-yl-propenone | 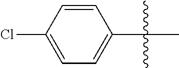 [SMILES: *c1ccc(Cl)cc1] | 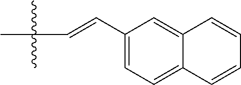 [SMILES: */C=C/c1ccc2ccccc2c1] |
| 134 | 1-[3-(4-Chloro-phenyl)- adamantan-1-yl]-3-(6- chloro-pyridin-3-yl)-propenone | 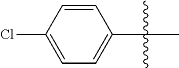 [SMILES: *c1ccc(Cl)cc1] | 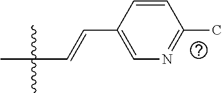 [SMILES: */C=C/c1ccc(C)nc1] |
| 135 | 1-[3-(4-Chloro-phenyl)- adamantan-1-yl]-3-(1H- imidazol-4-yl)-propenone | 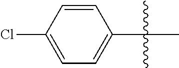 [SMILES: *c1ccc(Cl)cc1] | 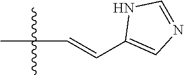 [SMILES: */C=C/c1cnc[nH]1] |
| 136 | 1-[3-(4-Chloro-phenyl)- adamantan-1-yl]-3-(9- ethyl-9H-carbazol- 3-yl)-propenone | 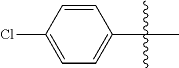 [SMILES: *c1ccc(Cl)cc1] | 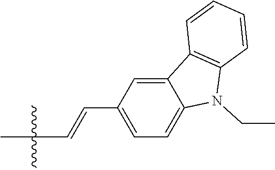 [SMILES: */C=C/c1ccc2c(c1)c1ccccc1n2CC] |
| 137 | 1-[3-(4-Chloro-phenyl)- adamantan-1-yl]-3-(4- phenyl-thiophen- 2-yl)-propenone | 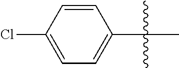 [SMILES: *c1ccc(Cl)cc1] | 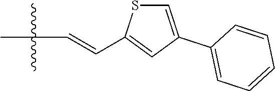 [SMILES: */C=C/c1cc(-c2ccccc2)cs1] |

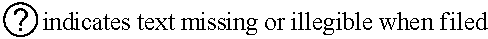

In various such embodiments, the compound is
3-(4-Chloro-phenyl)-adamantane-1-carboxylic acid phenylamide;
3-(4-Chloro-phenyl)-adamantane-1-carboxylic acid (4-hydroxy-phenyl)-amide;
Acetic acid 4-{[3-(4-chloro-phenyl)-adamantane-1-carbonyl]-amino}-phenyl ester;
3-(4-Chloro-phenyl)-adamantane-1-carboxylic acid (2,4-dihydroxy-phenyl)-amide;
3-(4-Chloro-phenyl)-adamantane-1-carboxylic acid (3-hydroxymethyl-phenyl)-amide;
3-(4-Chloro-phenyl)-adamantane-1-carboxylic acid (4-cyanomethyl-phenyl)-amide;
3-(4-Chloro-phenyl)-adamantane-1-carboxylic acid benzylamide;
3-(4-Chloro-phenyl)-adamantane-1-carboxylic acid 4-tert-butyl-benzylamide;
3-(4-Chloro-phenyl)-adamantane-1-carboxylic acid 4-methylsulfanyl-benzylamide;
3-(4-Chloro-phenyl)-adamantane-1-carboxylic acid 3-trifluoromethyl-benzylamide;
3-(4-Chloro-phenyl)-adamantane-1-carboxylic acid 4-trifluoromethyl-benzylamide;
3-(4-Chloro-phenyl)-adamantane-1-carboxylic acid 3,5-bis-trifluoromethyl-benzylamide;
3-(4-Chloro-phenyl)-adamantane-1-carboxylic acid 3-fluoro-5-trifluoromethyl-benzylamide;
3-(4-Chloro-phenyl)-adamantane-1-carboxylic acid 2-fluoro-4-trifluoromethyl-benzylamide;
3-(4-Chloro-phenyl)-adamantane-1-carboxylic acid 3,5-difluoro-benzylamide;
3-(4-Chloro-phenyl)-adamantane-1-carboxylic acid 3,4-difluoro-benzylamide;
3-(4-Chloro-phenyl)-adamantane-1-carboxylic acid 3,4,5-trifluoro-benzylamide;
3-(4-Chloro-phenyl)-adamantane-1-carboxylic acid 3-chloro-4-fluoro-benzylamide;
3-(4-Chloro-phenyl)-adamantane-1-carboxylic acid 4-fluoro-3-trifluoromethyl-benzylamide;
3-(4-Chloro-phenyl)-adamantane-1-carboxylic acid 2-chloro-4-fluoro-benzylamide;
3-(4-Chloro-phenyl)-adamantane-1-carboxylic acid 4-chloro-3-trifluoromethyl-benzylamide;
3-(4-Chloro-phenyl)-adamantane-1-carboxylic acid 3-aminomethyl-2,4,5,6-tetrachloro-benzylamide;
3-(4-Chloro-phenyl)-adamantane-1-carboxylic acid [1-(4-chloro-phenyl)-ethyl]-amide;
3-(4-Chloro-phenyl)-adamantane-1-carboxylic acid [1-(4-bromo-phenyl)-ethyl]-amide;
3-(4-Chloro-phenyl)-adamantane-1-carboxylic acid 4-methanesulfonyl-benzylamide;
3-(4-Chloro-phenyl)-adamantane-1-carboxylic acid 4-dimethylamino-benzylamide;
3-(4-Chloro-phenyl)-adamantane-1-carboxylic acid 4-trifluoromethoxy-benzylamide;
3-(4-Chloro-phenyl)-adamantane-1-carboxylic acid 3-trifluoromethoxy-benzylamide;
3-(4-Chloro-phenyl)-adamantane-1-carboxylic acid 4-phenoxy-benzylamide;
3-(4-Chloro-phenyl)-adamantane-1-carboxylic acid 3,4-dihydroxy-benzylamide;
3-(4-Chloro-phenyl)-adamantane-1-carboxylic acid phenethyl-amide;
3-(4-Chloro-phenyl)-adamantane-1-carboxylic acid [2-(4-fluoro-phenyl)-ethyl]-amide;
3-(4-Chloro-phenyl)-adamantane-1-carboxylic acid [2-(4-bromo-phenyl)-ethyl]-amide;
3-(4-Chloro-phenyl)-adamantane-1-carboxylic acid [2-(4-hydroxy-phenyl)-ethyl]-amide;
3-(4-Chloro-phenyl)-adamantane-1-carboxylic acid 4-phenoxy-benzylamide;
3-(4-Chloro-phenyl)-adamantane-1-carboxylic acid [2-(3-bromo-4-methoxy-phenyl)-ethyl]-amide;
3-(4-Chloro-phenyl)-adamantane-1-carboxylic acid [2-(3,4-dihydroxy-phenyl)-ethyl]-amide;
3-(4-Chloro-phenyl)-adamantane-1-carboxylic acid (2-benzo[1,3]dioxol-5-yl-ethyl)-amide;
3-(4-Chloro-phenyl)-adamantane-1-carboxylic acid [2-(3-phenoxy-phenyl)-ethyl]-amide;
3-(4-Chloro-phenyl)-adamantane-1-carboxylic acid [2-(4-phenoxy-phenyl)-ethyl]-amide;
3-(4-Chloro-phenyl)-adamantane-1-carboxylic acid (3-phenyl-propyl)-amide;
3-(4-Chloro-phenyl)-adamantane-1-carboxylic acid (biphenyl-4-ylmethyl)-amide;
3-(4-Chloro-phenyl)-adamantane-1-carboxylic acid (1-methyl-piperidin-4-yl)-amide;
3-(4-Chloro-phenyl)-adamantane-1-carboxylic acid (4-methyl-piperazin-1-yl)-amide;
3-(4-Chloro-phenyl)-adamantane-1-carboxylic acid (3-pyrrolidin-1-yl-propyl)-amide;
3-(4-Chloro-phenyl)-adamantane-1-carboxylic acid [3-(2-oxo-pyrrolidin-1-yl)-propyl]-amide;
3-(4-Chloro-phenyl)-adamantane-1-carboxylic acid [2-(1-methyl-pyrrolidin-2-yl)-ethyl]-amide;
3-(4-Chloro-phenyl)-adamantane-1-carboxylic acid (2-morpholin-4-yl-ethyl)-amide;
3-(4-Chloro-phenyl)-adamantane-1-carboxylic acid (2-piperazin-1-yl-ethyl)-amide;
3-(4-Fluoro-phenyl)-adamantane-1-carboxylic acid (pyridin-4-ylmethyl)-amide;
3-(4-Chloro-phenyl)-adamantane-1-carboxylic acid (pyridin-4-ylmethyl)-amide;
3-(4-Chloro-phenyl)-adamantane-1-carboxylic acid (2-pyridin-4-yl-ethyl)-amide;
3-(4-Chloro-phenyl)-adamantane-1-carboxylic acid (3-imidazol-1-yl-propyl)-amide;
3-(4-Chloro-phenyl)-adamantane-1-carboxylic acid (2-methyl-TH-indol-5-yl)-amide;
3-(4-Chloro-phenyl)-adamantane-1-carboxylic acid (1H-tetrazol-5-yl)-amide;
3-(4-Chloro-phenyl)-adamantane-1-carboxylic acid (9-ethyl-9H-carbazol-3-yl)-amide;
3-(4-Chloro-phenyl)-adamantane-1-carboxylic acid [4-(4-chloro-phenyl)-thiazol-2-yl]-amide;
3-(4-Chloro-phenyl)-adamantane-1-carboxylic acid benzothiazol-2-ylamide;
3-(4-Chloro-phenyl)-adamantane-1-carboxylic acid (5-chloro-benzooxazol-2-yl)-amide;
3-(4-Chloro-phenyl)-adamantane-1-carboxylic acid (9H-purin-6-yl)-amide;
3-(4-Chloro-phenyl)-adamantane-1-carboxylic acid (4-hydroxyphenyl)-amide;
[3-(4-Chloro-phenyl)-adamantan-1-ylmethyl]-(4-trifluoromethyl-benzyl)-amine;
[3-(4-Chloro-phenyl)-adamantan-1-ylmethyl]-(2-fluoro-4-trifluoromethyl-benzyl)-amine;
[3-(4-Chloro-phenyl)-adamantan-1-ylmethyl]-(4-fluoro-3-trifluoromethyl-benzyl)-amine;
[3-(4-Chloro-phenyl)-adamantan-1-ylmethyl]-(4-trifluoromethoxy-benzyl)-amine;
[3-(4-Chloro-phenyl)-adamantan-1-ylmethyl]-[2-(3-phenoxy-phenyl)-ethyl]-amine;
[3-(4-Chloro-phenyl)-adamantan-1-ylmethyl]-(1-methyl-piperidin-4-yl)-amine;
[3-(4-Chloro-phenyl)-adamantan-1-ylmethyl]-(4-methyl-piperazin-1-yl)-amine;
[3-(4-Chloro-phenyl)-adamantan-1-ylmethyl]-(3-pyrrolidin-1-yl-propyl)-amine;
[3-(4-Chloro-phenyl)-adamantan-1-ylmethyl]-[2-(1-methyl-pyrrolidin-2-yl)-ethyl]-amine;
[3-(4-Chloro-phenyl)-adamantan-1-ylmethyl]-(2-morpholin-4-yl-ethyl)-amine;
[3-(4-Chloro-phenyl)-adamantan-1-ylmethyl]-pyridin-4-ylmethyl-amine;
[3-(4-Chloro-phenyl)-adamantan-1-ylmethyl]-(9-ethyl-9H-carbazol-3-yl)-amine;
[3-(4-Chloro-phenyl)-adamantan-1-ylmethyl]-[5-(4-chloro-phenyl)-thiazol-2-yl]-amine;
Phenyl-[1-(3-phenyl-adamantan-1-yl)-ethyl]-amine;
{1-[3-(4-Fluoro-phenyl)-adamantan-1-yl]-ethyl}-phenyl-amine;
{1-[3-(4-Chloro-phenyl)-adamantan-1-yl]-ethyl}-phenyl-amine;
Benzyl-[1-(3-phenyl-adamantan-1-yl)-ethyl]-amine;
Benzyl-{1-[3-(4-fluoro-phenyl)-adamantan-1-yl]-ethyl}-amine;
Benzyl-{1-[3-(4-chloro-phenyl)-adamantan-1-yl]-ethyl}-amine;
(4-tert-Butyl-benzyl)-{1-[3-(4-chloro-phenyl)-adamantan-1-yl]-ethyl}-amine;
[1-(4-Bromo-phenyl)-ethyl]-{1-[3-(4-chloro-phenyl)-adamantan-1-yl]-ethyl}-amine;
[2-(4-Bromo-phenyl)-ethyl]-{1-[3-(4-chloro-phenyl)-adamantan-1-yl]-ethyl}-amine;
(1-Methyl-piperidin-4-yl)-[1-(3-phenyl-adamantan-1-yl)-ethyl]-amine;
{1-[3-(4-Fluoro-phenyl)-adamantan-1-yl]-ethyl}-(1-methyl-piperidin-4-yl)-amine;
{1-[3-(4-Chloro-phenyl)-adamantan-1-yl]-ethyl}-(1-methyl-piperidin-4-yl)-amine;
{1-[3-(4-Chloro-phenyl)-adamantan-1-yl]-ethyl}-(4-methyl-piperazin-1-yl)-amine;
{1-[3-(Phenyl)-adamantan-1-yl]-ethyl}-pyridin-4-ylmethyl-amine;
{1-[3-(4-Chloro-phenyl)-adamantan-1-yl]-ethyl}-(6-chloro-pyridin-3-ylmethyl)-amine;
{1-[3-(4-Chloro-phenyl)-adamantan-1-yl]-ethyl}-(2-pyridin-4-yl-ethyl)-amine;
{1-[3-(4-Chloro-phenyl)-adamantan-1-yl]-ethyl}-(3H-imidazol-4-ylmethyl)-amine;
{1-[3-(4-Chloro-phenyl)-adamantan-1-yl]-ethyl}-(2-methyl-1H-indol-5-yl)-amine;
{1-[3-(4-Chloro-phenyl)-adamantan-1-yl]-ethyl}-(9-ethyl-9H-carbazol-3-yl)-amine;
{1-[3-(4-Chloro-phenyl)-adamantan-1-yl]-ethyl}-(9-ethyl-9H-carbazol-3-ylmethyl)-amine;
9-Ethyl-9H-carbazole-3-carboxylic acid {1-[3-(4-chloro-phenyl)-adamantan-1-yl]-ethyl}-amide;
(4-Bromo-thiophen-2-ylmethyl)-{1-[3-(4-chloro-phenyl)-adamantan-1-yl]-ethyl}-amine;
{1-[3-(4-Chloro-phenyl)-adamantan-1-yl]-ethyl}-(4-phenyl-thiophen-2-ylmethyl)-amine;
   or a pharmaceutically acceptable salt or solvate (e.g., hydrate) thereof.

In various such embodiments, the compound is a compound of the formula

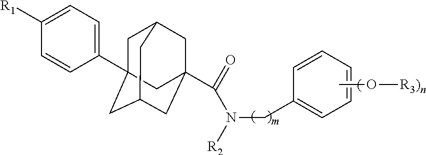

or a pharmaceutically acceptable salt or solvate (e.g., hydrate) thereof, wherein:
R₁ is H, Cl or F;
R₂ is H or alkyl;
m is 1 or 2;
n is 1, 2, 3, 4 or 5; and
each R₃ is independently H, —C(O)alkyl, —C(O)CH₂CH₂C(O)OH, R₄, —C(O)NR₅R₆, —P(O)(OR₇)₂ or glucosyl, provided that at least one R₃ is not H,
wherein
R₄ is a natural or unnatural amino acid linked through the carboxyl moiety as an ester,
R₅ is H or alkyl,
R₆ is H or alkyl, and
each R₇ is independently H or alkyl.

In various such embodiments, R₁ is Cl.

In various such embodiments, R₂ is H.

In various such embodiments, m is 2.

In various such embodiments, n is 1 or 2.

In various such embodiments, n is 2.

In various such embodiments, the

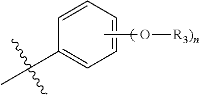

moiety has the structure

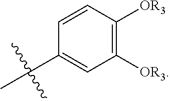

In various such embodiments, each R₃ is a —C(O)alkyl.

In various such embodiments, the

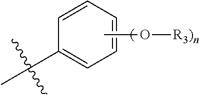

moiety has the structure

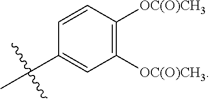

In various such embodiments, the compound is
acetic acid 2-acetoxy-5-(2-{[3-(4-chlorophenyl)-adamantane-1-carbonyl]-amino}ethyl)phenyl ester;
propionic acid 2-propionyloxy-5-(2-{[3-(4-chlorophenyl)-adamantane-1-carbonyl]-amino}ethyl)phenyl ester;
butyric acid 2-butyryloxy-5-(2-{[3-(4-chlorophenyl)-adamantane-1-carbonyl]-amino}ethyl)phenyl ester;
isobutyric acid 5-(2-{[3-(4-chlorophenyl)adamantane-1-carbonyl]amino}ethyl)-2-hydroxyphenyl ester; and
2-amino-3-methyl-butyric acid 5-(2-{[3-(4-chlorophenyl)adamantane-1-carbonyl]amino}ethyl)-2-hydroxyphenyl ester,
   or a salt or solvate thereof.

In various embodiments, the compound is acetic acid 2-acetoxy-5-(2-{[3-(4-chlorophenyl)-adamantane-1-carbonyl]-amino}ethyl)phenyl ester or a salt or solvate thereof.

Synthetic procedures and other information about these compounds is provided in International Patent Application Publications nos. 2006/138660 and 2010/105183, each of which is hereby incorporated herein by reference in its entirety.

In various embodiments of the methods, compositions and uses described herein, the compound is provided in the form of a pharmaceutical composition comprising the compound in combination with a pharmaceutically acceptable carrier, medium, or auxiliary agent.

In various embodiments of the methods, compositions and uses described herein, the compound is provided in combination or sequentially with an effective amount of at least one additional weight-control drug. For example, in various embodiments, the additional weight-control drug comprises bupropion-naltrexone, orlistat, phentermine-topiramate.

In various embodiments of the methods, compositions and uses described herein, wherein the compound is provided in combination or sequentially with an effective amount of at least one GLP-1 agonist. For example, in various embodiments wherein the GLP-1 agonist is liraglutide, semaglutide, lixisenatide, setmelanotide, exenatide, dulaglutide or tirzepatide.

In various embodiments of the methods, compositions and uses described herein, each aryl is a phenyl, naphthyl, 1,2,3,4-tetrahydronaphthalene, benzodioxole, or biphenyl.

In various embodiments of the methods, compositions and uses described herein, each aryl is a phenyl.

In various embodiments of the methods, compositions and uses described herein, each aryl (including phenyl) for which substitution is unspecified is optionally substituted by 1-4 groups each independently selected from halo, hydroxy, alkyl, haloalkyl, alkoxy, nitro, cyano, alkylamine, carboxy or alkoxycarbonyl.

In various embodiments of the methods, compositions and uses described herein, each aryl (including phenyl) for which substitution is unspecified is optionally substituted by 1-4 groups each independently selected from halo, alkyl, haloalkyl, hydroxy and alkoxy.

In various embodiments of the methods, compositions and uses described herein, each heteroaryl group is a 5-6-membered monocyclic heteroaryl having 1-3 heteroatoms selected from O, N and S.

In various embodiments of the methods, compositions and uses described herein, each heteroaryl group is an 8-10-membered bicyclic heteroaryl having 1-5 heteroatoms selected from O, N and S.

In various embodiments of the methods, compositions and uses described herein, each heteroaryl group is independently thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, benzopyrazolyl, purinyl, benzooxazolyl, or carbazolyl.

In various embodiments of the methods, compositions and uses described herein, each heteroaryl for which substitution is unspecified is optionally substituted by 1-4 groups each independently selected from halo, hydroxy, alkyl, haloalkyl, alkoxy, nitro, cyano, alkylamine, carboxy or alkoxycarbonyl.

In various embodiments of the methods, compositions and uses described herein, each heteroaryl for which substitution is unspecified is optionally substituted by 1-4 groups each independently selected from halo, alkyl, haloalkyl, hydroxy and alkoxy.

In various embodiments of the methods, compositions and uses described herein, each heterocycloalkyl group has from 3 to 7 members and includes 1-4 heteroatoms selected from O, N and S.

In various embodiments of the methods, compositions and uses described herein, each heterocycloalkyl group is independently piperidyl, piperazinyl, morpholinyl, pyrrolidinyl, thiomorpholinyl, thiazolidinyl, 1,3-dioxolanyl, 1,4-dioxanyl, tetrahydrofuranyl, tetrahydrothiophenyl, tetrahydrothiopyranyl, dihydrothienyl, dihydropyranyl, dihydrofuryl, or dihydrothiazolyl.

In various embodiments of the methods, compositions and uses described herein, each alkoxy for which a number of carbon atoms is not indicated independently has 1-6 carbon atoms, e.g., 1-4 carbon atoms, or 1-2 carbon atoms.

In various embodiments of the methods, compositions and uses described herein, each alkyl for which a number of carbon atoms is not indicated independently has 1-6 carbon atoms, e.g., 1-4 carbon atoms, or 1-2 carbon atoms.

The Examples, which follow, are illustrative of specific embodiments of the disclosure, and various uses thereof. They are set forth for explanatory purposes only, and are not to be taken as limiting the disclosure.

### EXAMPLES

The treatments of the disclosure may be better understood with reference to the following examples. These examples are intended to be representative of specific embodiments of the disclosure and are not intended as limiting the scope of the disclosure.

### Example 1

Opaganib suppresses HFD-induced body weight gain. Male C57BL/6J mice (8-week-old, 20-25 g) were purchased from Jackson Laboratories (Bar Harbor, ME) and housed under standard conditions. After acclimation for 1 week, mice were placed on a high-fat diet (HFD) in which 60% of calories are derived from fat (D12492 from Research Diets, New Brunswick, NJ). Control Diet (CD) mice received standard rodent chow which contains 5% crude fat. HFD mice were randomized into treatment groups receiving Vehicle (0.375% Tween in PBS) or 100 mg/kg Opaganib by oral gavage (0.1 ml volume) once per day 5-days per week (Monday-Friday) for up to 16 weeks. Similarly, CD mice were administered 0 (Vehicle) or 100 mg/kg Opaganib by gavage once per day 5-days per week. Food and water were provided ad libitum, and food consumption was monitored by weighing the chow remaining for each cage twice weekly. The health of each mouse was assessed daily, and mice were weighed twice per week.

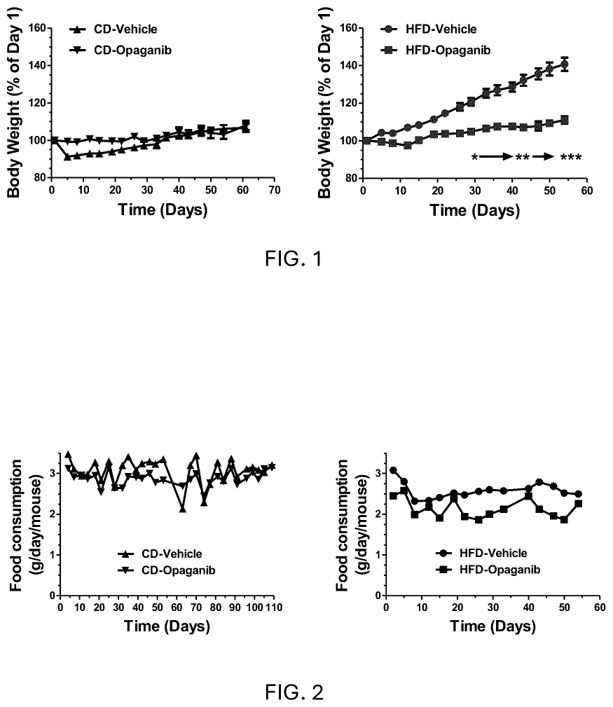

As shown in FIG. 1, mice given the control diet (CD) experienced a slight loss of body weight when the study was initiated but recovered to maintain essentially the starting body weight throughout the 8-week period. Treatment with 100 mg/kg opaganib did not affect body weights of the CD-fed mice. In contrast, vehicle-treated mice given a high-fat diet (HFD) progressively increased body weight, averaging a 41% gain by Week 8 (FIG. 1). In contrast, opaganib-treated mice on the HFD increased body weight by only 11% by Week 8, with the difference becoming increasingly more statistically significant over time (p<0.001 at Day 54).

### Example 2

Opaganib reduces food consumption. Food consumption was measured twice weekly and normalized for the number of mice in each cage. As shown in FIG. 2, mice treated with opaganib and fed the CD consumed 6% less than vehicle-treated controls (p<0.005) over the course of the experiment. Interestingly, vehicle-treated mice fed the HFD ate less food than vehicle-treated CD mice even though they had much greater increases in body weight. Over the course of the experiment, opaganib-treated mice ate 17% less HFD than the vehicle-treated controls (p<0.0001).

### Example 3

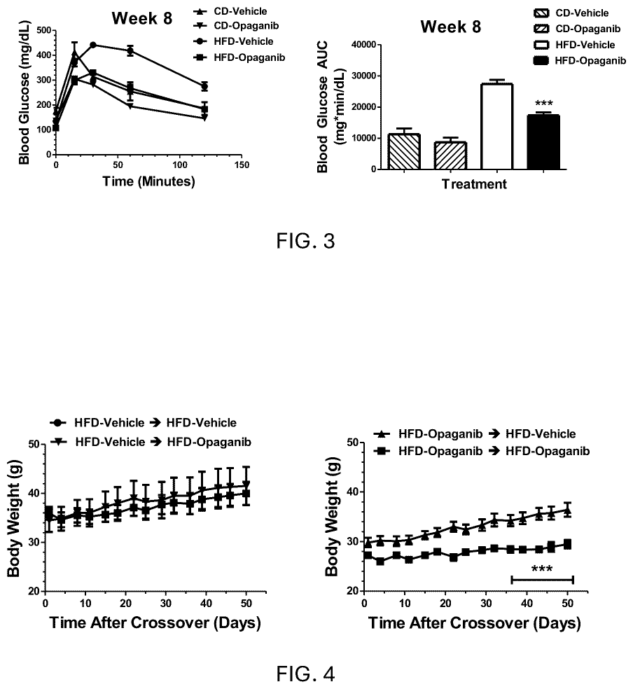

Opaganib improves HFD-induced loss of glucose tolerance. To assess the effects of HFD and opaganib on glucose metabolism, glucose tolerance tests were performed. Mice were fed either HFD or CD and treated with either vehicle or 100 mg/kg opaganib daily 5 days/week for 8 weeks. To determine fasting blood glucose levels, chow was removed overnight (12 hr) and drinking water was continued to be provided ad libitum. In the morning, 2 μL of blood was obtained from the tails following a lancet prick and analyzed using a Metene TD-4116 glucose monitoring system. For glucose tolerance testing, mice were administered a volume of 7.5×body weight (in kg) of a 25% glucose solution in water (w/v) via intraperitoneal injection. Tail blood was then collected at 15, 30, 60 and 120 minutes after the glucose injection and blood glucose levels were determined. Glucose concentrations were then plotted, and the area under the curve (AUC) for the plasma glucose level increase above baseline was calculated using GraphPad Prism 5.0. As shown in FIG. 3, the mean fasting blood glucose levels of HFD-fed mice were slightly lower than fasting glucose levels in CD mice (135 and 178 mg/dL, respectively). Fasting blood glucose levels in CD-fed mice and HFD-fed mice treated with opaganib were 23% and 20% lower than vehicle-treated mice on the same diet. HFD-Vehicle mice had substantially poorer glucose tolerance than did HFD-Opaganib mice, with AUCs of 27,387 and 17,339 mg*min/dL, respectively (37% decrease with opaganib, p<0.001). Additionally, CD-fed mice given opaganib also demonstrated 23% lower AUCs compared to CD-Vehicle mice, indicating the opaganib increases glucose clearance even in the absence of the HFD-challenge. Overall, HFD-feeding resulted in loss of glucose tolerance within 8 weeks, and opaganib-treatment reduced the fasting blood glucose levels and substantially improved glucose tolerance in HFD-fed mice.

### Example 4

Opaganib reduces weight gain by obese HFD-fed mice. To assess the ability of opaganib to improve body weight and glucose tolerance in mice already obese from HFD feeding, subsets of mice were crossed over to the opposite treatment protocol after 8 weeks on HFD. Consequently, the experimental groups after crossover included: HFD-Vehicle→Vehicle; HFD-Vehicle→Opaganib; HFD-Opaganib→Vehicle and HFD-Opaganib→Opaganib. Mice were maintained and monitored for an additional 8 weeks. As shown in FIG. 4, mice that were obese from 8 weeks of HFD-Vehicle further increased body weight when maintained on HFD and given either Vehicle or Opaganib for an additional 8 weeks (20.4% and 11.1% increase, respectively). Non-obese HFD-Opaganib mice gained substantial body weight when opaganib treatment was removed compared with mice that continued to receive opaganib (22.1% and 8.1% increases, respectively). Thus, opaganib treatment suppressed HFD-induced body weight gain through the entire 16-week period, resulting in average body weights of 41.5 and 29.5 g for HFD-Vehicle and HFD-Opaganib mice.

### Example 5

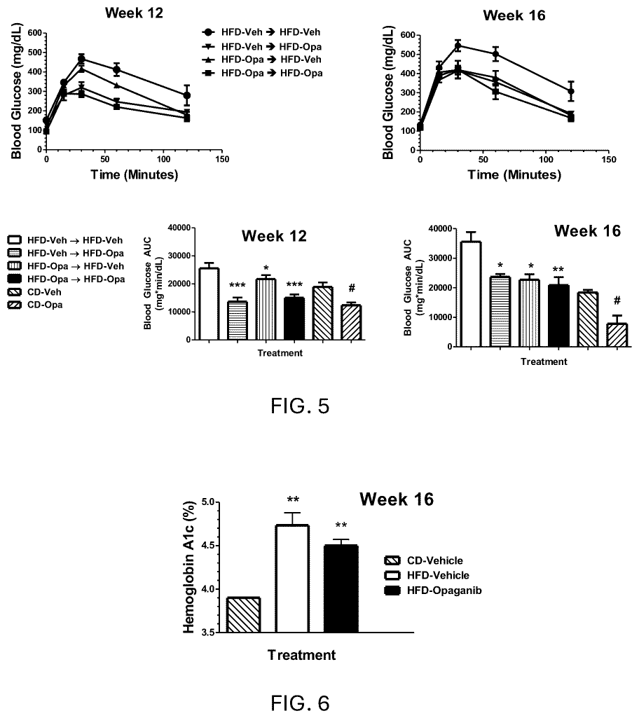

Opaganib restores glucose tolerance to HFD-fed mice. Glucose tolerance tests were performed at Week 12 and Week 16, i.e. at 4 weeks and 8 weeks after crossover to the opposite treatment. As shown in FIG. 5, the HFD-Vehicle→HFD-Vehicle group had very similar glucose kinetics and AUCs at Week 12 as HFD-Vehicle mice at Week 8. However, the glucose exposure substantially increased at the Week 16 time point indicating further loss of glucose control as the mice continued on the HFD. Similarly, the HFD-Opaganib→HFD-Opaganib mice had markedly better glucose tolerance and AUCs than the HFD-Vehicle mice at both 12 and 16 weeks (41% decreases at both time points). Importantly, mice that received HFD-Vehicle for 8 weeks and then crossed over to HFD-Opaganib had much better glucose kinetics and AUCs (47% and 33% decreases at 12 and 16 weeks, respectively) than mice that continued receiving HFD-Vehicle after the crossover point. This indicates that opaganib normalizes glucose clearance even in obese mice that had previously demonstrated impaired glucose tolerance in spite of maintaining the excess body weight accumulated in the first 8 weeks. This models the human clinical situation in which an obese diabetic patient would be treated with opaganib with the goal of improving their glucose tolerance even if their poor diets are maintained. Further improvement in glucose tolerance and potentially in weight loss might be observed if the mice/patients transition to a normal-fat diet at the time of opaganib treatment. Interestingly, mice in the HFD-Opaganib→HFD-Vehicle treatment group maintained significantly improved glucose tolerance compared with HFD-Vehicle→HFD-Vehicle mice for at least 8 weeks after removal of the opaganib treatment. Therefore, opaganib provides an unexpected sustained improvement in glucose tolerance even after removal of the drug.

### Example 6

Opaganib improves long-term glucose control in HFD-fed mice. Hemoglobin A1c (HbA1c) levels were measured at 16 weeks to assess the effects of HFD and opaganib on long-term glucose control. HbA1c levels were determined from 5 μL of tail blood using an A1CNOW Self Check system monitor (PTS Diagnostics, Whitestown, IN). As shown in FIG. 6, HbA1c levels in mice fed CD were all below the lower limit of detection (4%); whereas HbA1c in HFD-Vehicle animals were slightly, but significantly elevated (p<0.01). The modest increase in HbA1c is consistent with the lack of increases in fasting blood glucose levels in HFD-Vehicle mice (133 mg/dL at 16 weeks) and indicates that this HFD-induced obesity model does not fully represent the clinical manifestations of human diabetes. Nonetheless, HFD-fed mice treated with opaganib had slightly reduced (p=0.17) levels of HbA1c consistent with the lower fasting blood glucose concentrations (118 mg/dL at 16 weeks).

### Example 7

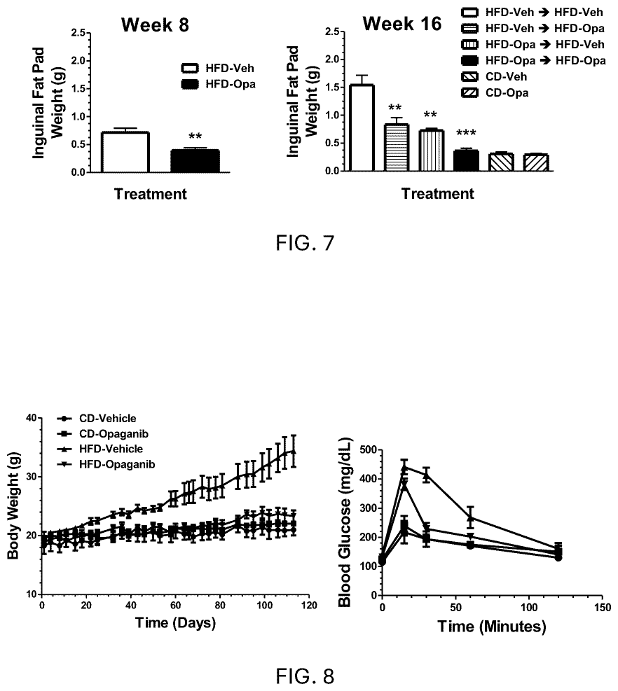

Opaganib reduces fat deposition in HFD-fed mice. At 8 or 16 weeks, mice were euthanized, and inguinal fat pads were collected. As shown in FIG. 7, the fat pads of HFD-Vehicle mice at both Week 8 and Week 16 were heavier than those harvested from CD-fed mice at Week 16. Treatment of CD-fed mice with opaganib did not affect fat pad weight at Week 16. In contrast, treatment of the HFD-fed mice with opaganib significantly reduced inguinal fat pad weight at Week 8 (p<0.01). For mice sacrificed at Week 16, the HFD-Vehicle→HFD-Vehicle group had much larger fat pads than did HFD-Opaganib→HFD-Opaganib mice (p<0.001) consistent with the overall lower body weights of the latter group. Importantly, crossover to HFD-Opaganib from HFD-Vehicle at Week 8 substantially reduced fat pad weight at Week 16, consistent with the improvement in glucose tolerance when these HFD-obese mice were treated with opaganib. Interestingly, HFD-fed mice that were initially treated with opaganib for 8 weeks and then with vehicle for an additional 8 weeks gained only small fat pad weight compared to mice that remained on opaganib treatment. This is consistent with the persistence of glucose control after opaganib is removed from HFD-fed mice (FIG. 5).

### Example 8

Opaganib also suppresses HFD-induced body weight gain in female mice. Because sex differences can occur in experimental models, we conducted similar HFD-induced obesity experiments using age-matched female C57BL/6J mice. As with male mice, a subgroup of HFD-Vehicle fed female mice was crossed over to HFD-Opaganib after 8 weeks of treatment. As expected, the starting body weights for female mice were substantially lower than those of male mice (19.0 and 25.3 g, respectively). At 8 and 16 weeks, body weights of female mice on CD increased by 9.3% and 14.9%, respectively; whereas, body weights of mice on HFD increased by 29.0% and 78.9% (FIG. 8). Opaganib-treatment reduced the gains in body weight at 8 and 16 weeks to 3.9% and 12.4% for mice on CD (p<0.001 compared to Vehicle), and 13.4% and 23.3% for mice on HFD (p<0.001 compared to Vehicle). As with male mice, the females ate significantly less HFD (2.2 g/mouse/day) than CD (3.0 g/mouse/day) (p<0.0001), and opaganib further reduced HFD consumption (p=0.0133). Thus, opaganib treatment caused prolonged suppression of female mouse weight gain similar to results with male mice.

### Example 9

Opaganib also suppresses HFD-induced loss of glucose tolerance in female mice. Glucose tolerance was substantially impaired in the female mice as early as 4 weeks of HFD (FIG. 8). Interestingly, the female mice on CD cleared the bolus of glucose much faster than did male mice on CD (FIG. 3) resulting in greater fold-increases in blood glucose AUCs for HFD-fed mice compared with those on CD. Nonetheless, as with male mice, opaganib-treatment substantially reduced the blood glucose AUCs in the HFD-fed female mice over the course of 16 weeks (Table 1). Additionally, opaganib treatment of obese female mice started after 8 weeks of HFD substantially reduced the blood glucose AUCs after 8 weeks on drug. Therefore, as with male mice, the glucose tolerance of obese female mice can be markedly improved by treatment with opaganib.

TABLE 1
Effects of opaganib on blood glucose AUCs in female mice. Female C57BL/6J mice were
given a CD or HFD and treated with either Vehicle or 100 mg/kg Opaganib once daily,
5 days/week. A subgroup of HFD-Vehicle mice were switched to opaganib treatment after
8 weeks (HFD-Opaganib). Glucose tolerance tests were administered after 4, 8, 12
and 16 weeks of treatment. Values indicated the mean ± SEM AUCs (mg*min/dL).

| Group | Week 4 | Week 8 | Week 12 | Week 16 |
|---|---|---|---|---|
| CD-Vehicle | 6,412 ± 1,719 | 4,372 ± 2,133 | 7,892 ± 384 | 11,230 ± 4,275 |
| CD-Opaganib | 6,425 ± 770 | 5,078 ± 1,228 | 6,674 ± 2,437 | 9,094 ± 2,525 |
| HFD-Vehicle | 17,261 ± 1,597 | 16,100 ± 1,303 | 23,703 ± 3,258 | 26,260 ± 2,747 |
| HFD-Opaganib | 9,550 ± 1,639 ** | 11,924 ± 1,803 | 14,344 ± 2,275* | 12,090 ± 2,450*** |
| HFD-Vehicle | NA | NA | 21,070 ± 885 | 16,230 ± 1,407* |
| → HFD- | | | | |
| Opaganib | | | | |

*p < 0.05,
** p < 0.01 and p < 0.001 for HFD-Opaganib compared with HFD-Vehicle.
NA—not applicable.

### Example 10

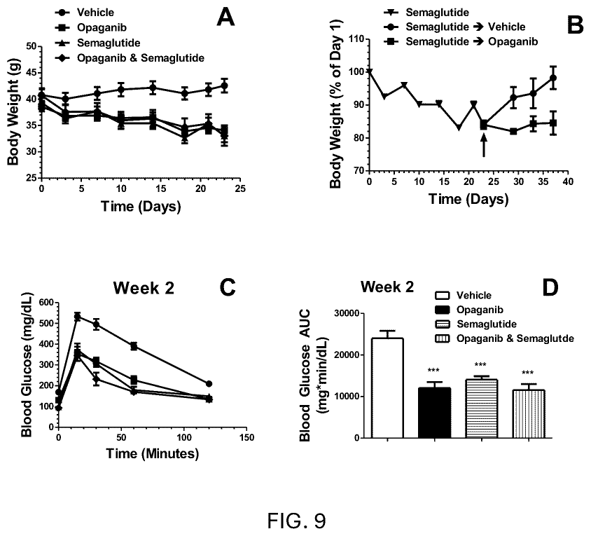

Opaganib can be combined with semaglutide to induce weight loss in obese mice. Obese male mice (16 or 18 weeks of HFD) were randomized into groups treated with Vehicle, opaganib alone, semaglutide alone or opaganib+semaglutide 5 days/week and body weights were monitored. As shown in FIG. 9A, HFD-fed mice treated with Vehicle continued to increase body weight (4.4% increase from Day 1 to Day 23); whereas mice treated with either opaganib or semaglutide lost body weight with changes of -11.7% and -15.8% relative to Day 1, respectively (p<0.001 for each treatment). Mice that received a combination of opaganib plus semaglutide had no apparent adverse effects and lost slightly more weight (−18.7%) than either opaganib alone or semaglutide alone. Interestingly, when treatment was paused each weekend, mice receiving semaglutide alone consistently increased body weight when measured on Monday (FIGS. 9A and 9B). In contrast, mice treated with opaganib or opaganib plus semaglutide did not demonstrate this body weight rebound during the “drug holiday” period. On Day 23, mice treated with semaglutide were randomized into groups for further treatment with either Vehicle or opaganib for an additional 2 weeks. As shown in FIG. 9B, mice previously treated with semaglutide rapidly gained body weight when treated with Vehicle; whereas, mice treated with opaganib maintained the weight reductions achieved by semaglutide treatment (p<0.01 compared with Vehicle group).

### Example 11

Opaganib can be combined with semaglutide to improve glucose tolerance in obese mice. Obese mice described in Example 10 were tested for glucose tolerance after 2 weeks of treatment with Vehicle, opaganib alone, semaglutide alone or opaganib plus semaglutide. As also shown in FIG. 9, glucose tolerance was substantially improved as early as 2 weeks of treatment with opaganib, semaglutide or the combination (p<0.001 for all treatment groups compared to Vehicle). Therefore, opaganib and semaglutide are both effective in promoting body weight loss in this model, and concurrent or sequential combination of the two drugs may provide increased therapeutic responses.

### Discussion

Although many studies have examined the roles of sphingolipids in obesity and related diseases, the accumulated information does not provide clarity on the expected effects of pharmacological manipulation of sphingolipid signaling. It is particularly unknown how a drug that inhibits multiple enzymes in the sphingolipid metabolism pathway will affect the development and progression of obesity and related pathologies. To the present inventors' knowledge, opaganib is the only compound described in the patent or journal literature that inhibits all three of SphK2, DES1 and GCS. Therefore, studies utilizing other inhibitors or genetic knockouts are not predictive of biological responses to opaganib because they do not replicate the multitargeting nature of opaganib. Additionally, the combined effects of simultaneous inhibition of multiple targets cannot be predicted by administration of multiple drugs because of differences in the absorption, metabolism and/or elimination of the individual drugs. In contrast, the multitargeting ability opaganib results in parallel inhibition of SphK2, DES1 and GCS. Furthermore, the combined effects of simultaneous inhibition of multiple targets cannot be predicted by genetic ablation of individual enzymes. Indeed, many proteins have non-enzymatic activities that are lost by genetic knockout but not by pharmacologic inhibition. For example, SphK2 contains a BH3 domain which regulates cell apoptosis independent of the synthesis of S1P. H. Liu et al., J Biol Chem 2003, 278, (41), 40330-6.

Of particular relevance to the present application, literature reports of the roles of SphK2, DES1 and GCS in the pathologies of obesity and Type 2 diabetes are inconsistent. Regarding SphK2, several studies suggest that its inhibition would be beneficial for patients with obesity/Type 2 diabetes, whereas others indicate that inhibition of SphK2 would be deleterious for such patients. For example, several groups have shown lower weight gain and reduced glucose intolerance in SphK2^{-/-} mice fed a HFD compared with normal mice. See, e.g., C. D. Green et al., Mol Metab 2024, 86, 101971; S. Ravichandran et al., Biochim Biophys Acta Mol Basis Dis 2019, 1865, (3), 570-576; Z. Song et al., FASEB J 2019, 33, (3), 3636-3646; J. Zhao et al., Int J Endocrinol Metab 2023, 21, (3), e136539. Additionally, Shi et al. showed that the purported SphK2 inhibitor 145 reduces hyperglycemia and indicators of non-alcoholic fatty liver disease in ob/ob mice. Y. Shi et al., Biochem Biophys Res Commun 2021, 580, 1-6. In contrast, Lee et al. showed that genetic overexpression of SphK2 in mice fed a HFD decreases glucose intolerance and insulin resistance. S. Y. Lee et al., Biochem Biophys Res Commun 2021, 580, 1-6. Also, Yuan et al. showed that siRNA against SphK2 or opaganib increases gluconeogenesis by hepatocytes and prevents the ability of insulin to suppress glucose production. J. Yuan et al, Arch Med Res 2018, 49, (5), 335-341. Further, Aji et al. showed that siRNA against SphK2 or treatment with opaganib suppresses insulin receptor endocytosis and recycling, and insulin-stimulated AKT phosphorylation in hepatocytes. G. Aji et al., Arch Med Res 2018, 49, (5), 335-341. Finally, Aji et al. showed that liver-specific knockout of SphK2 promotes insulin resistance and glucose intolerance. G. Aji et al., Proc Natl Acad Sci USA 2020, 117, (39), 24434-24442.

Similar inconsistencies exist in data on the role of DES1 in obesity and Type 2 diabetes. For example, Hu et al. demonstrated that knockdown of DES1 protects against palmitate-induced insulin resistance. W. Hu et al., J Biol Chem 2011, 286, (19), 16596-605. Additionally, Chaurasia et al. demonstrated that knockout of DES1 improves hepatic steatosis and insulin resistance in mice caused by leptin deficiency or HFD. B. Chaurasia et al., Science 2019, 365, (6451), 386-392. Finally, Veeriah et al. demonstrated that the putative DES1 inhibitor N-trans-caffeoyltyramine decreases weight gain and hepatic steatosis in HFD-fed mice. V. Veeriah et al., Cell Death Dis 2022, 13, (1), 89. In opposition to these studies, Zarini et al. demonstrated that serum levels of dihydroceramides are higher in patients with Type 2 diabetes than controls, and that treatment of myotubes in vitro with dihydroceramides decreases insulin sensitivity. S. Zarini et al., J Lipid Res 2022, 63, (10), 100270. Similarly, Denimal et al. demonstrated that plasma levels of dihydroceramides are higher in patients with Type 2 diabetes than controls, and that treatment of Type 2 diabetes patients with the GLP-1 receptor agonist liraglutide reduces plasma dihydroceramide levels and decreases liver fat content. D. Denimal et al., Cardiovasc Diabetol 2023, 22, (1), 104. Also, Barbarorja et al. demonstrated the DES1 is suppressed in diabetic patients, and this impairs adipocyte differentiation. N. Barbarroja et al., Diabetes 2015, 64, (4), 1180-92. Finally, Rustamov et al. demonstrated that the putative DES1 inhibitor GT-11 reduces glucose uptake by myotubes. PLoS One 2010, 5, (6), e11239.

Similar uncertainty exists for the role of GCS in obesity/Type 2 diabetes. For example, Bijl et al. demonstrated that the putative GCS inhibitor AMP-DNM restores insulin signaling in the liver and corrects blood glucose values in ob/ob mice. N. Also, Van Eijk et al. demonstrated that the putative GCS inhibitor AMP-DNM restores insulin signaling in adipocytes isolated from leptin-deficient obese mice. M. van Eijk et al., PLoS One 2009, 4, (3), e4723. Furthermore, Zhao et al. demonstrated that the putative GCS inhibitor MZ-21 improves glucose homeostasis and reduces hepatic steatosis in ob/ob mice. H. Zhao et al., Hepatology 2009, 50, (1), 85-93. Additionally, Yew et al. demonstrated that the putative GCS inhibitor Genz-112638 increases glucose tolerance and decreases HbA1c in HFD-fed mice, and Richards et al. demonstrated that the putative GCS inhibitor EXEL-0346 increases glucose tolerance in HFD-fed mice. N. S. Yew et al., PLoS One 2010, 5, (6), e11239; S. Richards et al., J Med Chem 2012, 55, (9), 4322-35. Lastly, Jang et al. demonstrated that the putative GCS inhibitor PDMP reduces adipocytes formation in HFD-fed mice. H. J. Jang et al., FASEB J 2020, 34, (1), 1270-1287. On the other hand, several studies have demonstrated that administration of glucosylceramide to mice, rats or humans improves glucose tolerance and/or decreases liver fat. See, e.g., T. Adar et al., Clin Exp Hepatol 2023, 9, (2), 164-171; G. Lalazar et al., J Med Food 2017, 20, (5), 458-464; M. Margalit et al., J Pharmacol Exp Ther 2006, 319, (1), 105-10; E. Zigmond et al., J Inflamm Res 2014, 7, 151-8.; E. Zigmond et al., Am J Physiol Endocrinol Metab 2009, 296, (1), E72-8.

Overall, the contradictory results discussed above for each of the target enzymes for opaganib make it impossible to predict the therapeutic effects of this drug in the development and progression of obesity and Type 2 diabetes. Therefore, the effects of opaganib in an established model of obesity and Type 2 diabetes was unpredictable, in the absence of the data provided herein.

All patents, patent applications, and published references cited herein are hereby incorporated by reference in their entirety. Various modifications and variations of the described compositions, uses and disclosure the disclosure will be apparent to those skilled in the art without departing from the scope and spirit of the disclosure. Although the treatments of the disclosure have been described in connection with specific embodiments, it will be understood that the disclosure should not be unduly limited to such specific embodiments. Indeed, various modifications of the described modes for carrying out the treatments of the disclosure that are obvious to those skilled in the fields of molecular biology, medicine, immunology, pharmacology, virology, or related fields are intended to be within the scope of the disclosure.

Various aspects of the disclosure are provided by the following enumerated embodiments, which may be combined in any number and in any combination not technically or logically inconsistent.
Embodiment 1. A method for suppressing the appetite for food comprising administering to a subject a compound in an amount effective to inhibit SphK2, DES1 and/or GCS.
Embodiment 2. A method for suppressing the appetite for food, the method comprising administering to a subject an effective amount of a compound.
Embodiment 3. A composition for use in suppressing the appetite for food, the composition including a compound in an amount effective to inhibit SphK2, DES1 and/or GC.
Embodiment 4. A composition for use in suppressing the appetite for food, the composition including an effective amount of a compound.
Embodiment 5. Use of a compound in an amount effective to inhibit SphK2, DES1 and/or GCS for suppressing the appetite for food.
Embodiment 6. Use of a compound for suppressing the appetite for food.
Embodiment 7. A method for promoting weight loss comprising administering to a subject a compound in an amount effective to inhibit SphK2, DES1 and/or GCS.
Embodiment 8. A method for promoting weight loss, the method comprising administering to a subject an effective amount of a compound.
Embodiment 9. A composition for use in promoting weight loss, the composition including a compound in an amount effective to inhibit SphK2, DES1 and/or GC.
Embodiment 10. A composition for use in promoting weight loss, the composition including an effective amount of a compound.
Embodiment 11. Use of a compound in an amount effective to inhibit SphK2, DES1 and/or GCS for promoting weight loss.
Embodiment 12. Use of a compound for promoting weight loss.
Embodiment 13. A method for preventing or treating obesity comprising administering to a patient a compound in an amount effective to inhibit SphK2, DES1 and/or GCS.
Embodiment 14. A method for preventing or treating obesity, the method comprising administering to a subject an effective amount of a compound.
Embodiment 15. A composition for use in preventing or treating obesity, the composition including a compound in an amount effective to inhibit SphK2, DES1 and/or GC.
Embodiment 16. A composition for use in preventing or treating obesity, the composition including an effective amount of a compound.
Embodiment 17. Use of a compound in an amount effective to inhibit SphK2, DES1 and/or GCS for preventing or treating obesity.
Embodiment 18. Use of a compound for preventing or treating obesity.
Embodiment 19. A method for preventing or treating an obesity-induced disease comprising administering to a patient a compound in an amount effective to inhibit SphK2, DES1 and/or GCS.
Embodiment 20. A method for preventing or treating an obesity-induced disease, the method comprising administering to a subject an effective amount of a compound.
Embodiment 21. A composition for use in preventing or treating an obesity-induced disease, the composition including a compound in an amount effective to inhibit SphK2, DES1 and/or GC.
Embodiment 22. A composition for use in preventing or treating an obesity-induced disease, the composition including an effective amount of a compound.
Embodiment 23. Use of a compound in an amount effective to inhibit SphK2, DES1 and/or GCS for preventing or treating an obesity-induced disease.
Embodiment 24. Use of a compound for preventing or treating an obesity-induced disease.
Embodiment 25. A method, composition or use of any of embodiments 13-18, wherein the obesity-induced disease is diabetes.
Embodiment 26. A method, composition or use of embodiment 19, wherein diabetes is not insulin-dependent diabetes mellitus.
Embodiment 27. A method, composition or use of any of embodiments 19-26, wherein the obesity-induced disease is metabolic syndrome, heart disease, gallbladder disease, high blood pressure, sleep apnea, kidney disease, stroke, fatty liver disease, high cholesterol, respiratory disease, depression, gout or infertility.
Embodiment 28. A method, composition or use of any of embodiments 19-27, wherein the obesity-induced disease is not any of cancer, inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity, including allergic encephalomyelitis, allergic neuritis, transplant allograft rejection, graft versus host disease, myocarditis, thyroiditis, nephritis, systemic lupus erythematosus, insulin-dependent diabetes mellitus, diabetic retinopathy, arthritis, psoriasis, Kaposi's sarcoma, hemangiomas, myocardial angiogenesis, atherosclerotic plaque neovascularization, choroidal neovascularization, retinopathy of prematurity (retrolental fibroplasias), macular degeneration, corneal graft rejection, rubeosis, neuroscular glaucoma and Oster Webber syndrome.
Embodiment 29. A method for treating Type 1 diabetes comprising administering to a patient a compound in an amount effective to inhibit SphK2, DES1 and/or GCS.
Embodiment 30. A method for preventing or treating Type 1 diabetes, the method comprising administering to a subject an effective amount of a compound.
Embodiment 31. A composition for use in preventing or treating Type 1 diabetes, the composition including a compound in an amount effective to inhibit SphK2, DES1 and/or GC.
Embodiment 32. A composition for use in preventing or treating Type 1 diabetes, the composition including an effective amount of a compound.
Embodiment 33. Use of a compound in an amount effective to inhibit SphK2, DES1 and/or GCS for preventing or treating Type 1 diabetes.
Embodiment 34. Use of a compound for preventing or treating Type 1 diabetes.
Embodiment 35. The method, composition for use, or use of any of embodiments 29-34, wherein the Type 1 diabetes is not insulin-dependent.
Embodiment 36. A method for treating Type 2 diabetes comprising administering to a patient a compound in an amount effective to inhibit SphK2, DES1 and/or GCS.
Embodiment 37. A method for preventing or treating Type 2 diabetes, the method comprising administering to a subject an effective amount of a compound.
Embodiment 38. A composition for use in preventing or treating Type 2 diabetes, the composition including a compound in an amount effective to inhibit SphK2, DES1 and/or GC.
Embodiment 39. A composition for use in preventing or treating Type 2 diabetes, the composition including an effective amount of a compound.
Embodiment 40. Use of a compound in an amount effective to inhibit SphK2, DES1 and/or GCS for preventing or treating Type 2 diabetes.
Embodiment 41. Use of a compound for preventing or treating Type 2 diabetes.
Embodiment 42. The method, composition for use, or use of any of embodiments 36-41, wherein the Type 1 diabetes is not insulin-dependent.
Embodiment 43. The method, composition or use of any of embodiments 1-42, wherein the compound is a is provided in an amount in an amount effective to inhibit SphK2.
Embodiment 44. The method, composition or use of any of embodiments 1-43, wherein the compound is provided in an amount effective to inhibit DES1.
Embodiment 45. The method, composition or use of any of embodiments 1-44, wherein the compound is provided in an amount effective to inhibit GCS.
Embodiment 46. The method, composition, or use of any of embodiments 1-42, wherein the compound is provided in an amount effective to inhibit two or more of SphK2, DES1 and GCS.
Embodiment 47. The method, composition, or use of any of embodiments 1-42, wherein the compound is provided in an amount effective to inhibit all three of SphK2, DES1 and GCS.
Embodiment 48. The method, composition or use of any of embodiments 1-47, wherein the compound is opaganib.
Embodiment 49. The method, composition or use of any of embodiments 1-47, wherein the compound is 3-(4-chlorophenyl)-N-(pyridin-4-ylmethyl)-1-adamantanecarboxamide or a pharmaceutically acceptable salt or hydrate thereof.
Embodiment 50. The method, composition or use of any of embodiments 1-47, wherein the compound is a compound as described in any of International Patent Application Publication no. 2006138660 and U.S. Pat. Nos. 8,063,248, 8,557,800, and RE49811.
Embodiment 51. The method, composition or use of any of embodiments 1-47, wherein the compound is a compound as described in any of International Patent Application Publication no. 2010105183 and U.S. Pat. No. 8,685,936.
Embodiment 52. The method, composition or use of any of embodiments 1-47, wherein the compound is a compound of the formula

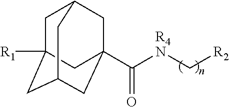

or a pharmaceutically acceptable salt or solvate (e.g., hydrate) thereof, wherein:
   R₁ is phenyl, 4-chlorophenyl or 4-fluorophenyl;
   R₂ is 4-pyridyl, optionally substituted with up to 4 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆) alkyl, and wherein each alkyl portion of a substituent is optionally further substituted with 1, 2, or 3 groups independently selected from halogen, CN, OH, and NH₂;
   R₄ is H or alkyl; and
   n is 1 or 2.
Embodiment 53. The method, composition or use of embodiment 52, wherein R₁ is 4-fluorophenyl or 4-chlorophenyl.
Embodiment 54. The method, composition or use of any of embodiments 1-47, wherein the compound is:
3-(4-fluorophenyl)-N-(pyridin-4-ylmethyl)adamantane-1-carboxamide;
3-(4-chlorophenyl)-N-(pyridin-4-ylmethyl)adamantane-1-carboxamide; or
3-(4-chlorophenyl)-N-(2-pyridin-4-ylethyl)adamantane-1-carboxamide,
   or a pharmaceutically acceptable salt or solvate (e.g., hydrate) thereof.
   Embodiment 55. The method, composition or use of any of embodiments 1-47, wherein the compound is a compound of the formula

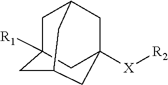

or a pharmaceutically acceptable salt or solvate (e.g., hydrate) thereof, wherein:
   X is —C(R₃,R₄)N(R₅)— or —C(O)N(R₄)—;
   R₁ is phenyl substituted with 1 to 5 groups that are independently halogen, haloalkyl, CONR′R″, —OC(O)NR′R″, —NR′C(O)R″, —CF₃, —OCF₃, —CN, —CO₂H, —S-alkyl, —SOR′R″, SO₂R′, wherein R′ and R″ are independently H or (C₁-C₆)alkyl, and wherein each alkyl portion of a substituent is optionally further substituted with 1, 2, or 3 groups independently selected from halogen, CN, OH, and NH₂;
   R₂ is aryl, -alkyl-aryl, heteroaryl, -alkyl-heteroaryl, heterocycloalkyl or -alkyl-heterocycloalkyl;
   R₃ is H or -alkyl;
   wherein the alkyl and ring portion of each of the above R₂ and R₃ groups is optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂; and
   R₄ and R₅ are independently H or alkyl.
Embodiment 56. The method, composition or use of embodiment 55, wherein X is —C(O)N(R₄)—.
Embodiment 57. The method, composition or use of embodiment 56, wherein R₄ is H.
Embodiment 58. The method, composition or use of any of embodiments 55-57, wherein X is —C(R₃,R₄)N(R₅)—.
Embodiment 59. The method, composition or use of embodiment 58, wherein R₃ is H or alkyl and R₄ is H.
Embodiment 60. The method, composition or use of any of embodiments 55-59, wherein R₁ is aryl (e.g., phenyl) substituted with one or two halogen groups.
Embodiment 61. The method, composition or use of any of embodiments 55-60, wherein R₂ is aryl (e.g., phenyl) or -alkyl-aryl (e.g., benzyl or phenethyl).
Embodiment 62. The method, composition or use of any of embodiments 55-60, wherein R₂ is phenyl, benzyl, phenethyl, or 3-phenylpropyl, in which the alkyl and ring portion of is optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂.
Embodiment 63. The method, composition or use of any of embodiments 55-60, wherein R₂ is heterocycloalkyl or -alkyl-heterocycloalkyl.
Embodiment 64. The method, composition or use of embodiment 63, wherein each heterocycloalkyl is independently selected from a piperidinyl, a piperazinyl, a pyrrolidinyl, and a morpholinyl.
Embodiment 65. The method, composition or use of any of embodiments 55-60, wherein R₂ is piperidinyl, piperazinyl, 2-(piperazin-1-yl)ethyl, 2(pyrrolidin-2-yl)ethyl, 3-(pyrrolidin-1-yl)propyl, or 2-(morpholin-4-yl)ethyl, in which the alkyl and ring portion of the above R₂ groups is optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂.
Embodiment 66. The method, composition or use of any of embodiments 55-60, wherein R₂ is heteroaryl or -alkyl-heteroaryl.
Embodiment 67. The method, composition or use of embodiment 66, wherein each heteroaryl is independently selected from pyridinyl (e.g., pyridin-4-yl or pyridin-3-yl), indoyl (e.g., 1H-indol-5-yl), tetrazolyl (e.g., 1H-tetrazol-5-yl), imidazolyl (e.g., 3H-imidazol-4-y), benzothiazolyl (e.g., benzothiazol-2-yl), carbazolyl (e.g., carbazol-3-yl), benzooxazolyl (e.g., benzooxazol-2-yl), purinyl (e.g., purin-6-yl) and thienyl (e.g., thien-2-yl).
Embodiment 68. The method, composition or use of any of embodiments 55-60, wherein R₂ is pyridin-4-ylmethyl, pyridin-4-ylethyl, pyridin-3-ylmethyl, 1H-indol-5-yl, 1H-tetrazol-5-yl, 3H-imidazol-4-ylmethyl, benzothiazol-2-yl, carbazol-3-yl, benzooxazol-2-yl, purin-6-yl, or thiophen-2-yl, in which the alkyl and ring portion of the above R₂ groups is optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or —NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂.
Embodiment 69. The method, composition or use of any of embodiments 1-47, wherein the compound is:
3-(4-Chloro-phenyl)-adamantane-1-carboxylic acid phenylamide;
3-(4-Chloro-phenyl)-adamantane-1-carboxylic acid (4-hydroxy-phenyl)-amide;
Acetic acid 4-{[3-(4-chloro-phenyl)-adamantane-1-carbonyl]-amino}-phenyl ester;
3-(4-Chloro-phenyl)-adamantane-1-carboxylic acid (2,4-dihydroxy-phenyl)-amide;
3-(4-Chloro-phenyl)-adamantane-1-carboxylic acid (3-hydroxymethyl-phenyl)-amide;
3-(4-Chloro-phenyl)-adamantane-1-carboxylic acid (4-cyanomethyl-phenyl)-amide;
3-(4-Chloro-phenyl)-adamantane-1-carboxylic acid benzylamide;
3-(4-Chloro-phenyl)-adamantane-1-carboxylic acid 4-tert-butyl-benzylamide;
3-(4-Chloro-phenyl)-adamantane-1-carboxylic acid 4-methylsulfanyl-benzylamide;
3-(4-Chloro-phenyl)-adamantane-1-carboxylic acid 3-trifluoromethyl-benzylamide;
3-(4-Chloro-phenyl)-adamantane-1-carboxylic acid 4-trifluoromethyl-benzylamide;
3-(4-Chloro-phenyl)-adamantane-1-carboxylic acid 3,5-bis-trifluoromethyl-benzylamide;
3-(4-Chloro-phenyl)-adamantane-1-carboxylic acid 3-fluoro-5-trifluoromethyl-benzylamide;
3-(4-Chloro-phenyl)-adamantane-1-carboxylic acid 2-fluoro-4-trifluoromethyl-benzylamide;
3-(4-Chloro-phenyl)-adamantane-1-carboxylic acid 3,5-difluoro-benzylamide;
3-(4-Chloro-phenyl)-adamantane-1-carboxylic acid 3,4-difluoro-benzylamide;
3-(4-Chloro-phenyl)-adamantane-1-carboxylic acid 3,4,5-trifluoro-benzylamide;
3-(4-Chloro-phenyl)-adamantane-1-carboxylic acid 3-chloro-4-fluoro-benzylamide;
3-(4-Chloro-phenyl)-adamantane-1-carboxylic acid 4-fluoro-3-trifluoromethyl-benzylamide;
3-(4-Chloro-phenyl)-adamantane-1-carboxylic acid 2-chloro-4-fluoro-benzylamide;
3-(4-Chloro-phenyl)-adamantane-1-carboxylic acid 4-chloro-3-trifluoromethyl-benzylamide;
3-(4-Chloro-phenyl)-adamantane-1-carboxylic acid 3-aminomethyl-2,4,5,6-tetrachloro-benzylamide;
3-(4-Chloro-phenyl)-adamantane-1-carboxylic acid [1-(4-chloro-phenyl)-ethyl]-amide;
3-(4-Chloro-phenyl)-adamantane-1-carboxylic acid [1-(4-bromo-phenyl)-ethyl]-amide;
3-(4-Chloro-phenyl)-adamantane-1-carboxylic acid 4-methanesulfonyl-benzylamide;
3-(4-Chloro-phenyl)-adamantane-1-carboxylic acid 4-dimethylamino-benzylamide;
3-(4-Chloro-phenyl)-adamantane-1-carboxylic acid 4-trifluoromethoxy-benzylamide;
3-(4-Chloro-phenyl)-adamantane-1-carboxylic acid 3-trifluoromethoxy-benzylamide;
3-(4-Chloro-phenyl)-adamantane-1-carboxylic acid 4-phenoxy-benzylamide;
3-(4-Chloro-phenyl)-adamantane-1-carboxylic acid 3,4-dihydroxy-benzylamide;
3-(4-Chloro-phenyl)-adamantane-1-carboxylic acid phenethyl-amide;
3-(4-Chloro-phenyl)-adamantane-1-carboxylic acid [2-(4-fluoro-phenyl)-ethyl]-amide;
3-(4-Chloro-phenyl)-adamantane-1-carboxylic acid [2-(4-bromo-phenyl)-ethyl]-amide;
3-(4-Chloro-phenyl)-adamantane-1-carboxylic acid [2-(4-hydroxy-phenyl)-ethyl]-amide;
3-(4-Chloro-phenyl)-adamantane-1-carboxylic acid 4-phenoxy-benzylamide;
3-(4-Chloro-phenyl)-adamantane-1-carboxylic acid [2-(3-bromo-4-methoxy-phenyl)-ethyl]-amide;
3-(4-Chloro-phenyl)-adamantane-1-carboxylic acid [2-(3,4-dihydroxy-phenyl)-ethyl]-amide;
3-(4-Chloro-phenyl)-adamantane-1-carboxylic acid (2-benzo[1,3]dioxol-5-yl-ethyl)-amide;
3-(4-Chloro-phenyl)-adamantane-1-carboxylic acid [2-(3-phenoxy-phenyl)-ethyl]-amide;
3-(4-Chloro-phenyl)-adamantane-1-carboxylic acid [2-(4-phenoxy-phenyl)-ethyl]-amide;
3-(4-Chloro-phenyl)-adamantane-1-carboxylic acid (3-phenyl-propyl)-amide;
3-(4-Chloro-phenyl)-adamantane-1-carboxylic acid (biphenyl-4-ylmethyl)-amide;
3-(4-Chloro-phenyl)-adamantane-1-carboxylic acid (1-methyl-piperidin-4-yl)-amide;
3-(4-Chloro-phenyl)-adamantane-1-carboxylic acid (4-methyl-piperazin-1-yl)-amide;
3-(4-Chloro-phenyl)-adamantane-1-carboxylic acid (3-pyrrolidin-1-yl-propyl)-amide;
3-(4-Chloro-phenyl)-adamantane-1-carboxylic acid [3-(2-oxo-pyrrolidin-1-yl)-propyl]-amide;
3-(4-Chloro-phenyl)-adamantane-1-carboxylic acid [2-(1-methyl-pyrrolidin-2-yl)-ethyl]-amide;
3-(4-Chloro-phenyl)-adamantane-1-carboxylic acid (2-morpholin-4-yl-ethyl)-amide;
3-(4-Chloro-phenyl)-adamantane-1-carboxylic acid (2-piperazin-1-yl-ethyl)-amide;
3-(4-Fluoro-phenyl)-adamantane-1-carboxylic acid (pyridin-4-ylmethyl)-amide;
3-(4-Chloro-phenyl)-adamantane-1-carboxylic acid (pyridin-4-ylmethyl)-amide;
3-(4-Chloro-phenyl)-adamantane-1-carboxylic acid (2-pyridin-4-yl-ethyl)-amide;
3-(4-Chloro-phenyl)-adamantane-1-carboxylic acid (3-imidazol-1-yl-propyl)-amide;
3-(4-Chloro-phenyl)-adamantane-1-carboxylic acid (2-methyl-TH-indol-5-yl)-amide;
3-(4-Chloro-phenyl)-adamantane-1-carboxylic acid (1H-tetrazol-5-yl)-amide;
3-(4-Chloro-phenyl)-adamantane-1-carboxylic acid (9-ethyl-9H-carbazol-3-yl)-amide;
3-(4-Chloro-phenyl)-adamantane-1-carboxylic acid [4-(4-chloro-phenyl)-thiazol-2-yl]-amide;
3-(4-Chloro-phenyl)-adamantane-1-carboxylic acid benzothiazol-2-ylamide;
3-(4-Chloro-phenyl)-adamantane-1-carboxylic acid (5-chloro-benzooxazol-2-yl)-amide;
3-(4-Chloro-phenyl)-adamantane-1-carboxylic acid (9H-purin-6-yl)-amide;
3-(4-Chloro-phenyl)-adamantane-1-carboxylic acid (4-hydroxyphenyl)-amide;
[3-(4-Chloro-phenyl)-adamantan-1-ylmethyl]-(4-trifluoromethyl-benzyl)-amine;
[3-(4-Chloro-phenyl)-adamantan-1-ylmethyl]-(2-fluoro-4-trifluoromethyl-benzyl)-amine;
[3-(4-Chloro-phenyl)-adamantan-1-ylmethyl]-(4-fluoro-3-trifluoromethyl-benzyl)-amine;
[3-(4-Chloro-phenyl)-adamantan-1-ylmethyl]-(4-trifluoromethoxy-benzyl)-amine;
[3-(4-Chloro-phenyl)-adamantan-1-ylmethyl]-[2-(3-phenoxy-phenyl)-ethyl]-amine;
[3-(4-Chloro-phenyl)-adamantan-1-ylmethyl]-(1-methyl-piperidin-4-yl)-amine;
[3-(4-Chloro-phenyl)-adamantan-1-ylmethyl]-(4-methyl-piperazin-1-yl)-amine;
[3-(4-Chloro-phenyl)-adamantan-1-ylmethyl]-(3-pyrrolidin-1-yl-propyl)-amine;
[3-(4-Chloro-phenyl)-adamantan-1-ylmethyl]-[2-(1-methyl-pyrrolidin-2-yl)-ethyl]-amine;
[3-(4-Chloro-phenyl)-adamantan-1-ylmethyl]-(2-morpholin-4-yl-ethyl)-amine;
[3-(4-Chloro-phenyl)-adamantan-1-ylmethyl]-pyridin-4-ylmethyl-amine;
[3-(4-Chloro-phenyl)-adamantan-1-ylmethyl]-(9-ethyl-9H-carbazol-3-yl)-amine;
[3-(4-Chloro-phenyl)-adamantan-1-ylmethyl]-[5-(4-chloro-phenyl)-thiazol-2-yl]-amine;
Phenyl-[1-(3-phenyl-adamantan-1-yl)-ethyl]-amine;
{1-[3-(4-Fluoro-phenyl)-adamantan-1-yl]-ethyl}-phenyl-amine;
{1-[3-(4-Chloro-phenyl)-adamantan-1-yl]-ethyl}-phenyl-amine;
Benzyl-[1-(3-phenyl-adamantan-1-yl)-ethyl]-amine;
Benzyl-{1-[3-(4-fluoro-phenyl)-adamantan-1-yl]-ethyl}-amine;
Benzyl-{1-[3-(4-chloro-phenyl)-adamantan-1-yl]-ethyl}-amine;
(4-tert-Butyl-benzyl)-{1-[3-(4-chloro-phenyl)-adamantan-1-yl]-ethyl}-amine;
[1-(4-Bromo-phenyl)-ethyl]-{1-[3-(4-chloro-phenyl)-adamantan-1-yl]-ethyl}-amine;
[2-(4-Bromo-phenyl)-ethyl]-{1-[3-(4-chloro-phenyl)-adamantan-1-yl]-ethyl}-amine;
(1-Methyl-piperidin-4-yl)-[1-(3-phenyl-adamantan-1-yl)-ethyl]-amine;
{1-[3-(4-Fluoro-phenyl)-adamantan-1-yl]-ethyl}-(1-methyl-piperidin-4-yl)-amine;
{1-[3-(4-Chloro-phenyl)-adamantan-1-yl]-ethyl}-(1-methyl-piperidin-4-yl)-amine;
{1-[3-(4-Chloro-phenyl)-adamantan-1-yl]-ethyl}-(4-methyl-piperazin-1-yl)-amine;
{1-[3-(Phenyl)-adamantan-1-yl]-ethyl}-pyridin-4-ylmethyl-amine;
{1-[3-(4-Chloro-phenyl)-adamantan-1-yl]-ethyl}-(6-chloro-pyridin-3-ylmethyl)-amine;
{1-[3-(4-Chloro-phenyl)-adamantan-1-yl]-ethyl}-(2-pyridin-4-yl-ethyl)-amine;
{1-[3-(4-Chloro-phenyl)-adamantan-1-yl]-ethyl}-(3H-imidazol-4-ylmethyl)-amine;
{1-[3-(4-Chloro-phenyl)-adamantan-1-yl]-ethyl}-(2-methyl-1H-indol-5-yl)-amine;
{1-[3-(4-Chloro-phenyl)-adamantan-1-yl]-ethyl}-(9-ethyl-9H-carbazol-3-yl)-amine;
{1-[3-(4-Chloro-phenyl)-adamantan-1-yl]-ethyl}-(9-ethyl-9H-carbazol-3-ylmethyl)-amine;
9-Ethyl-9H-carbazole-3-carboxylic acid {1-[3-(4-chloro-phenyl)-adamantan-1-yl]-ethyl}-amide;
(4-Bromo-thiophen-2-ylmethyl)-{1-[3-(4-chloro-phenyl)-adamantan-1-yl]-ethyl}-amine;
{1-[3-(4-Chloro-phenyl)-adamantan-1-yl]-ethyl}-(4-phenyl-thiophen-2-ylmethyl)-amine;
   or a pharmaceutically acceptable salt or solvate (e.g., hydrate) thereof.
   Embodiment 70. The method, composition or use of any of embodiments 1-47, wherein the compound is a compound of the formula

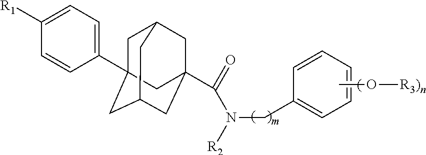

or a pharmaceutically acceptable salt or solvate (e.g., hydrate) thereof, wherein:
   R₁ is H, Cl or F;
   R₂ is H or alkyl;
   m is 1 or 2;
   n is 1, 2, 3, 4 or 5; and
   each R₃ is independently H, —C(O)alkyl, —C(O)CH₂CH₂C(O)OH, R₄, —C(O)NR₅R₆, —P(O)(OR₇)₂ or glucosyl, provided that at least one R₃ is not H,
   wherein
   R₄ is a natural or unnatural amino acid linked through the carboxyl moiety as an ester,
   R₅ is H or alkyl,
   R₆ is H or alkyl, and
   each R₇ is independently H or alkyl.
Embodiment 71. A method, composition or use of embodiment 70, wherein R₁ is Cl.
Embodiment 72. A method, composition or use of embodiment 70 or embodiment 71, wherein R₂ is H.
Embodiment 73. A method, composition or use of any of embodiments 70-72, wherein m is 2.
Embodiment 74. A method, composition or use of any of embodiments 70-73, wherein n is 1 or 2.
Embodiment 75. A method, composition or use of any of embodiments 70-73, wherein n is 2.
Embodiment 76. A method, composition or use of any of embodiments 70-75, wherein the

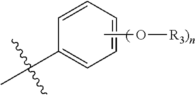

moiety has the structure

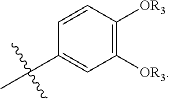

Embodiment 77. A method, composition or use of any of embodiments 70-76, wherein each R₃ is a —C(O)alkyl.
Embodiment 78. A method, composition or use of any of embodiments 70-77, wherein the

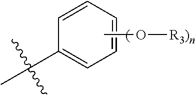

moiety has the structure

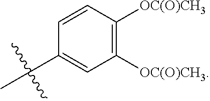

Embodiment 79. A method, composition or use of any of embodiments 1-47, wherein the compound is
acetic acid 2-acetoxy-5-(2-{[3-(4-chlorophenyl)-adamantane-1-carbonyl]-amino}ethyl)phenyl ester;
propionic acid 2-propionyloxy-5-(2-{[3-(4-chlorophenyl)-adamantane-1-carbonyl]-amino}ethyl)phenyl ester;
butyric acid 2-butyryloxy-5-(2-{[3-(4-chlorophenyl)-adamantane-1-carbonyl]-amino}ethyl)phenyl ester;
isobutyric acid 5-(2-{[3-(4-chlorophenyl)adamantane-1-carbonyl]amino}ethyl)-2-hydroxyphenyl ester; and
2-amino-3-methyl-butyric acid 5-(2-{[3-(4-chlorophenyl)adamantane-1-carbonyl]amino}ethyl)-2-hydroxyphenyl ester,
   or a salt or solvate thereof.
   Embodiment 80. A method, composition or use of any of embodiments 1-47, wherein the compound is acetic acid 2-acetoxy-5-(2-{[3-(4-chlorophenyl)-adamantane-1-carbonyl]-amino}ethyl)phenyl ester or a salt or solvate thereof.
   Embodiment 81. The method, composition or use of any of embodiments 1-80, wherein the compound is provided in the form of a pharmaceutical composition comprising the compound in combination with a pharmaceutically acceptable carrier, medium, or auxiliary agent.
   Embodiment 82. The method, composition or use of any of embodiments 1-81, wherein the compound is provided in combination or sequentially with an effective amount of at least one additional weight-control drug.
   Embodiment 83. The method, composition or use of embodiment 82, wherein the additional weight-control drug comprises bupropion-naltrexone, orlistat, phentermine-topiramate.
   Embodiment 84. The method, composition or use of any of embodiments 1-81, wherein the compound is provided in combination or sequentially with an effective amount of at least one GLP-1 agonist.
   Embodiment 85. The method, composition or use of embodiment 84, wherein the GLP-1 agonist is liraglutide, semaglutide, lixisenatide, setmelanotide, exenatide, dulaglutide or tirzepatide.
   Embodiment 86. The method, composition or use of any embodiment above, wherein each aryl is a phenyl, naphthyl, 1,2,3,4-tetrahydronaphthalene, benzodioxole, or biphenyl.
   Embodiment 87. The method, composition or use of any embodiment above, wherein each aryl is a phenyl.
   Embodiment 88. The method, composition or use of any embodiment above, wherein each aryl (including phenyl) for which substitution is unspecified is optionally substituted by 1-4 groups each independently selected from halo, hydroxy, alkyl, haloalkyl, alkoxy, nitro, cyano, alkylamine, carboxy or alkoxycarbonyl.
   Embodiment 89. The method, composition or use of any embodiment above, wherein each aryl (including phenyl) for which substitution is unspecified is optionally substituted by 1-4 groups each independently selected from halo, alkyl, haloalkyl, hydroxy and alkoxy.
   Embodiment 90. The method, composition or use of any embodiment above, wherein each heteroaryl group is a 5-6-membered monocyclic heteroaryl having 1-3 heteroatoms selected from O, N and S.
   Embodiment 91. The method, composition or use of any embodiment above but for embodiment 76, wherein each heteroaryl group is an 8-10-membered bicyclic heteroaryl having 1-5 heteroatoms selected from O, N and S.
   Embodiment 92. The method, composition or use of any embodiment above wherein each heteroaryl group is independently thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, benzopyrazolyl, purinyl, benzooxazolyl, or carbazolyl.
   Embodiment 93. The method, composition or use of any embodiment above, wherein each heteroaryl for which substitution is unspecified is optionally substituted by 1-4 groups each independently selected from halo, hydroxy, alkyl, haloalkyl, alkoxy, nitro, cyano, alkylamine, carboxy or alkoxycarbonyl.
   Embodiment 94. The method, composition or use of any embodiment above, wherein each heteroaryl for which substitution is unspecified is optionally substituted by 1-4 groups each independently selected from halo, alkyl, haloalkyl, hydroxy and alkoxy.
   Embodiment 95. The method, composition or use of any embodiment above, wherein each heterocycloalkyl group has from 3 to 7 members and includes 1-4 heteroatoms selected from O, N and S.
   Embodiment 96. The method, composition or use of any embodiment above, wherein each heterocycloalkyl group is independently piperidyl, piperazinyl, morpholinyl, pyrrolidinyl, thiomorpholinyl, thiazolidinyl, 1,3-dioxolanyl, 1,4-dioxanyl, tetrahydrofuranyl, tetrahydrothiophenyl, tetrahydrothiopyranyl, dihydrothienyl, dihydropyranyl, dihydrofuryl, or dihydrothiazolyl.
   Embodiment 97. The method, composition or use of any embodiment above, wherein each alkoxy for which a number of carbon atoms is not indicated independently has 1-6 carbon atoms, e.g., 1-4 carbon atoms, or 1-2 carbon atoms.
   Embodiment 98. The method, composition or use of any embodiment above, wherein each alkyl for which a number of carbon atoms is not indicated independently has 1-6 carbon atoms, e.g., 1-4 carbon atoms, or 1-2 carbon atoms.

We claim:

1. A method for
suppressing the appetite for food,
promoting weight loss,
preventing or treating obesity,
preventing or treating an obesity-induced disease,
preventing or treating Type 1 diabetes, or
preventing or treating Type 2 diabetes
the method comprising administering to a subject a compound in an amount effective to inhibit SphK2, DES1 and/or GCS.

2. The method of claim 1, wherein the compound is 3-(4-chlorophenyl)-N-(pyridin-4-ylmethyl)-1-adamantanecarboxamide or a pharmaceutically acceptable salt or hydrate thereof.

3. The method of claim 1, wherein the compound is a compound of the formula 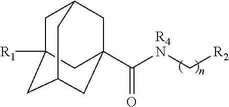
or a pharmaceutically acceptable salt or solvate (e.g., hydrate) thereof, wherein:
R₁ is phenyl, 4-chlorophenyl or 4-fluorophenyl;
R₂ is 4-pyridyl, optionally substituted with up to 4 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, and NH₂;
R₄ is H or alkyl; and
n is 1 or 2.

4. The method of claim 1, wherein the compound is a compound of the formula 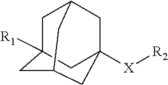
or a pharmaceutically acceptable salt or solvate (e.g., hydrate) thereof, wherein:
X is —C(R₃,R₄)N(R₅)— or —C(O)N(R₄)—;
R₁ is phenyl substituted with 1 to 5 groups that are independently halogen, haloalkyl, CONR′R″, —OC(O)NR′R″, —NR′C(O)R″, —CF₃, —OCF₃, —CN, —CO₂H, —S-alkyl, —SOR′R″, SO₂R′, wherein R′ and R″ are independently H or (C₁-C₆)alkyl, and wherein each alkyl portion of a substituent is optionally further substituted with 1, 2, or 3 groups independently selected from halogen, CN, OH, and NH₂;
R₂ is aryl, -alkyl-aryl, heteroaryl, -alkyl-heteroaryl, heterocycloalkyl or -alkyl-heterocycloalkyl;
R₃ is H or -alkyl;
wherein the alkyl and ring portion of each of the above R₂ and R₃ groups is optionally substituted with up to 5 groups that are independently (C₁-C₆)alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆)alkyl, and wherein each alkyl portion of a substituent is optionally further substituted with 1, 2, or 3 groups independently selected from halogen, CN, OH, and NH₂; and
R₄ and R₅ are independently H or alkyl.

5. The method, of claim 1, wherein the compound is a compound of the formula 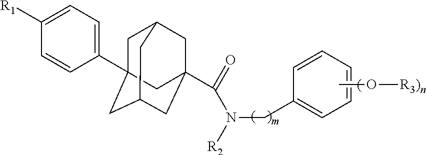
or a pharmaceutically acceptable salt or solvate (e.g., hydrate) thereof, wherein:
R₁ is H, Cl or F;
R₂ is H or alkyl;
m is 1 or 2;
n is 1, 2, 3, 4 or 5; and
each R₃ is independently H, —C(O)alkyl, —C(O)CH₂CH₂C(O)OH, R₄, —C(O)NR₅R₆, —P(O)(OR₇)₂ or glucosyl, provided that at least one R₃ is not H,
wherein
R₄ is a natural or unnatural amino acid linked through the carboxyl moiety as an ester,
R₅ is H or alkyl,
R₆ is H or alkyl, and
each R₇ is independently H or alkyl.

6. The method of claim 1, wherein the method is for suppressing the appetite for food.

7. The method of claim 1, wherein the method is for promoting weight loss.

8. The method of claim 1, wherein the method is for preventing or treating obesity.

9. The method of claim 1, wherein the method is for preventing or treating an obesity-induced disease.

10. The method of claim 8, wherein the obesity-induced disease is diabetes.

11. The method of claim 8, wherein the obesity-induced disease is not diabetes.

12. The method of claim 8, wherein the obesity-induced disease is metabolic syndrome, heart disease, gallbladder disease, high blood pressure, sleep apnea, kidney disease, stroke, fatty liver disease, high cholesterol, respiratory disease, depression, gout or infertility.

13. The method of claim 8, wherein the obesity-induced disease is not any of cancer, inflammatory bowel disease, arthritis, atherosclerosis, asthma, allergy, inflammatory kidney disease, circulatory shock, multiple sclerosis, chronic obstructive pulmonary disease, skin inflammation, periodontal disease, psoriasis and T cell-mediated diseases of immunity, including allergic encephalomyelitis, allergic neuritis, transplant allograft rejection, graft versus host disease, myocarditis, thyroiditis, nephritis, systemic lupus erythematosus, insulin-dependent diabetes mellitus, diabetic retinopathy, arthritis, psoriasis, Kaposi's sarcoma, hemangiomas, myocardial angiogenesis, atherosclerotic plaque neovascularization, choroidal neovascularization, retinopathy of prematurity (retrolental fibroplasias), macular degeneration, corneal graft rejection, rubeosis, neuroscular glaucoma and Oster Webber syndrome.

14. The method of claim 1, wherein the method is for preventing or treating Type 1 diabetes.

15. The method of claim 1, wherein the method is for preventing or treating Type 2 diabetes.

16. The method of claim 1, wherein the method is for preventing Type 1 or Type 2 diabetes that is not insulin-dependent.

17. The method of claim 1, wherein the compound is a is provided in an amount in an amount effective to inhibit SphK2.

18. The method of claim 1, wherein the compound is provided in an amount effective to inhibit DES1.

19. The method of claim 1, wherein the compound is provided in an amount effective to inhibit GCS.

20. The method of claim 1, wherein the compound is provided in combination or sequentially with an effective amount of at least one additional drug selected from bupropion-naltrexone, orlistat, phentermine-topiramate, liraglutide, semaglutide, lixisenatide, setmelanotide, exenatide, dulaglutide and tirzepatide.
